(19) United States
(12) Patent Application Publication
(10) Pub. No.: US 2025/0295762 A1
(43) Pub. Date: Sep. 25, 2025

(54) COMPOSITE BIOACTIVE COMPOSITIONS AND APPLICATIONS THEREOF

(71) Applicant: THE UNIVERSITY OF NORTH CAROLINA AT CHAPEL HILL, Chapel Hill, NC (US)

(72) Inventors: Kristy Ainslie, Chapel Hill, NC (US); Eric Bachelder, Chapel Hill, NC (US); Cole Batty, Chapel Hill, NC (US); Liubov Lifshits, Chapel Hill, NC (US); John Andrew Roque, III, Chapel Hill, NC (US)

(21) Appl. No.: 18/848,823

(22) PCT Filed: Mar. 21, 2023
(86) PCT No.: PCT/US2023/015745

§ 371 Date: Sep. 19, 2024

Related U.S. Application Data
(60) Provisional application No. 63/322,017, filed on Mar. 21, 2022.

(51) Int. Cl. A61K 39/385; A61K 39/00; A61K 47/02; A61K 47/22; A61K 47/36; A61K 47/42

(52) U.S. Cl. CPC A61K 39/385; A61K 47/02; A61K 47/22; A61K 47/36; A61K 47/42; A61K 2039/6093; A61K 2039/64

(57) ABSTRACT

In one aspect, composite compositions are described herein for delivery of various bioactive compositions. In some embodiments, a composite composition comprises a metal-organic coordination polymer matrix, and one or more bioactive compositions carried by the metal-organic coordination polymer matrix, wherein the metal-organic coordination polymer matrix comprises polymeric chains including a repeating unit of formula I.

### RELATED APPLICATION DATA

This application is a U.S. National Phase of PCT/US23/15745 filed Mar. 21, 2023, which claims priority pursuant to Article 8 of the Patent Cooperation Treaty to U.S. Provisional Patent Application Ser. No. 63/322,017 filed Mar. 21, 2022, each of which is hereby incorporated herein by reference in its entirety.

### STATEMENT REGARDING FEDERALLY SPONSORED RESEARCH OF DEVELOPMENT

This invention was made with government support under Grant Number 75N93019C00052 awarded by the National Institutes of Health. The government has certain rights in the invention.

### FIELD

The present invention relates to bioactive compositions and, in particular, to composite compositions wherein bioactive agents are carried by degradable metal-organic coordination polymers.

### BACKGROUND

Metal-based coordination polymers with organic ligands have been reported as 1-dimensional, 2D and 3D structures, with 2D and 3D structures containing internal pores and referred to as metal-organic frameworks (MOFs). Construction of MOFs often require harsh solvents (e.g., dimethylformamide, ethanol) and elevated temperature, which can denature biological molecular species, including proteins and nucleic acids. Moreover, MOFs can show resistance to biodegradation, thereby limiting the efficient release of therapeutic agents contained or carried therein.

### SUMMARY

In one aspect, the foregoing disadvantages are addressed by composite compositions described herein employing metal-organic coordination polymer matrices. In some embodiments, a composite composition comprises a metal-organic coordination polymer matrix, and one or more bioactive compositions carried by the metal-organic coordination polymer matrix, wherein the metal-organic coordination polymer matrix comprises polymeric chains including a repeating unit of formula I:

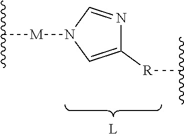

where M is a transition metal and L is a linker comprising a moiety for coordinating with the transition metal. R is a part of a chemical structure of the linker L, and R can include various functional groups capable of binding to the transition metal M.

Any moiety consistent with the technical objectives described herein can be used in the linker structure for coordination with a transition metal. In some embodiments, R comprises a moiety including an oxygen atom for coordinating with the transition metal. For example, R may comprise ether, hydroxyl, or carboxyl group. In other embodiments, R may comprise a moiety employing a nitrogen or sulfur for coordinating with the transition metal.

In some embodiments, the carboxyl and imidazole moieties for formula I are provided by histidine. The amine group of the histidine, in some embodiments, can be unsubstituted or substituted, as desired. The amine of histidine can form an amide bond with another amino acid, for example. The amino acid can be an α-amino acid, β-amino acid, or unnatural amino acid. The metal-organic coordination polymer below, for example, includes the histidine being functionalized with β-alanine.

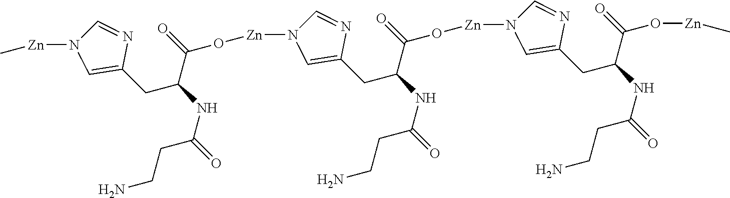

In this way, various peptides can be incorporated into the polymeric chains of the metal-organic coordination polymer matrix, as described further herein. Additionally, in some embodiments, various solvents can be coordinating to M.

In some embodiments, various substituents can be incorporated into the metal-organic coordination polymer matrix via the amine of the histidine. Such substituents can be used to vary properties of the metal-organic coordination polymer matrix. In some embodiments, the substituents can enhance hydrophilic or hydrophobic character of the metal-organic coordination polymer matrix. Moreover, substituents can be chosen to produce a favorable environment in the metal-organic coordination polymer matrix for the one or more bioactive compositions carried therein. In some embodiments, the organic species coordinating with metal centers is carnosine.

Any transition metal consistent with the technical objectives described herein can be used as metal centers in the metal-organic coordination polymer matrices. In some embodiments, M is a transition metal, such as zinc or other Group 10-12 transition metal of the Periodic Table. The transition metal M is a noble metal, in some embodiments. In some embodiments, the transition metal is a first row transition metal including, but not limited to, manganese, copper, and cobalt.

The bioactive composition can comprise any compound, pharmaceutical, biologic, nucleic acid, and/or protein. The bioactive composition can be natural or synthetic. The bioactive composition, for example, can be a naturally occurring protein or a recombinant protein. In some embodiments, the bioactive composition comprises one or more antigens. In such embodiments, composite compositions described herein can be employed in vaccine delivery. The bioactive composition, in other embodiments, can comprise one or more pharmaceutical compounds including, but not limited to, chemotherapeutics, antiviral compounds, and/or antimicrobial compounds. Specific identity of the bioactive composition can be dependent on several considerations including, but not limited to, the disease or indication being treated and interaction of the bioactive composition with the metal-organic coordination polymer matrix.

The bioactive composition can be present in the composite composition in any desired amount. In some embodiments, the bioactive composition is present in an amount up to 70 weight percent of the composite composition. The bioactive composition can also be present in an amount selected from Table I.

```
TABLE I
Amount of Bioactive Composition (wt. %)
0.5-50
0.5-20
0.5-10
  1-30
  1-15
```

As described herein, the bioactive composition is carried or supported by the metal-organic coordination polymer matrix. The bioactive composition can reside on surfaces of the metal-organic coordination polymer matrix and/or be encapsulated by the metal-organic coordination polymer matrix. In some embodiments, polymeric chains of the matrix are stacked and exhibit a fiber or fibrous morphology. The bioactive composition can reside on the fibers formed of the stacked polymeric chains and/or within spaces between the chains and/or fibers. In some embodiments, the metal-organic coordination polymer matrix can adopt a particle morphology, as described further herein.

The composite composition, in some embodiments, further comprises an adjuvant for the bioactive composition. Specific identity of the adjuvant will be dependent on the identity of the bioactive composition. In some embodiments, the adjuvant comprises one or more ligands including natural and/or synthetic nucleic acids, proteins, lipids, and sugars. In some embodiments, adjuvant ligands are agonists for pattern recognition receptors (PRR), including Toll-like receptors, NOD-like receptors, RIG-I-like receptors, STING, mast cell agonists, and C-type lectin receptors. Selected PRR ligands can be selected from Table 2, in some embodiments.

TABLE 2
Adjuvant Ligands

TLR2 Ligands
TLR3 and RLR Ligands
TLR4 Ligands
TLR5 Ligands
TLR7/8 Ligands
TLR9 Ligands
NOD2 Ligands
STING Ligands
RIG-I Ligands
Mast Cell Agonists

In some embodiments, the adjuvant is operable for promoting antigen presentation by the major histocompatibility complexes (MHC).

Adjuvant can be present in the composite composition in any desired amount. In some embodiments, adjuvant is present in an amount of 0.01-20 weight percent or 0.1-10 weight percent of the composite composition. Adjuvant can reside on surfaces of or be encapsulated in the metal-organic coordination polymer matrix. In some embodiments, adjuvant is adsorbed on surfaces of the metal-organic coordination polymer matrix.

In another embodiment, more than one bioactive agent can reside on the surface or be encapsulated in the metal-organic coordination polymer matrix. For example, an antigen and an adjuvant can be incorporated into the composite composition. In some embodiments, the bioactive agent is adsorbed on one or more surfaces of the metal-organic coordination polymer matrix. The adsorption of the bioactive agent can be physical adsorption or chemisorption.

The bioactive composition, in some embodiments, can be released from the composite composition via degradation of the metal-organic coordination polymer matrix. In some embodiments, degradation of the metal-organic coordination polymer matrix is pH dependent. Coordination between organic sections of the polymer and metal can be broken at certain pH values, leading to matrix degradation and release of the bioactive composition. For example, the imidazole nitrogen of formula I above can break coordination with the metal at pH below neutral pH (acidic pH). Accordingly, the metal-organic coordination polymer matrix can degrade and release the bioactive composition in environments where the pH is below neutral pH. Such environments can occur upon internalization of the composite composition by phagocytic cells (e.g., macrophages, dendritic cells (DCs)) and exposure to the acidic pH of the phagosome. Other acidic biological environments can also be employed for degradation of the metal-organic coordination polymer matrix and release of the bioactive composition.

In some embodiments, composite compositions described herein are suspended in a polysaccharide solution. The polysaccharide solution can comprise one or more polysaccharides. Any polysaccharide operable for the suspension of particles and/or other morphologies of composite compositions described herein can be employed. In some embodiments, particles of composite compositions are suspended in mannan solution. Mannan, for example, can be present in the solution in an amount of 0.1-5% w/v.

In another aspect, methods of treating patients are described herein. A method, in some embodiments, comprises providing a composite composition including a metal-organic coordination polymer matrix, and one or more bioactive compositions carried by the metal-organic coordination polymer matrix, wherein the metal-organic coordination polymer matrix comprises polymeric chains including a repeating unit of formula I.

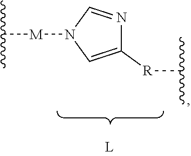

where M is a transition metal, L is a linker comprising a moiety for coordinating with the transition metal, and R is a part of a chemical structure of the linker L. As described above, R, in some embodiments, comprises a moiety including an oxygen atom for coordinating with the transition metal. The composite composition is administered to the patient. In some embodiments, the composition is injected into the patient, such as injected intramuscularly or another administration route. The composite composition can have any parameters, characteristics, architecture, and/or properties described herein. In some embodiments, for example, the composite composition comprises an antigen in conjunction with an adjuvant. The adjuvant comprises one or more ligands for pattern recognition receptors. The method further comprises releasing the antigen from the metal-organic coordination polymer matrix and eliciting an immunogenic response in the patient. In some embodiments, methods described herein are employed for vaccine delivery or administration.

These and other embodiments are further illustrated in the following non-limiting examples of the detailed description.

### DESCRIPTION OF THE FIGURES

FIG. 1A is a schematic of 3D-metal-organic framework (MOF) formation.

FIG. 1B is schematic of 1D metal-organic coordination polymer formation.

FIG. 1C shows the formation of Zinc-Carnosine (ZnCar) coordination polymer formed in neutral pH in HEPES buffer.

FIG. 2A is a scanning electron micrograph image of ZnCar.

FIG. 2B is a cryogenic electron micrograph of ZnCar displaying lattice fringes indicative of stacks of polymeric chains.

FIG. 2C includes a PXRD pattern of ZnCar as synthesized (ZnCar experimental), compared with PXRD pattern simulated from crystal structure of ZnCar-MOF (DMF solvate) reported in literature (see Katsoulidis, A. P.; Park, K. S.; Antypov, D.; Marti-Gastaldo, C.; Miller, G. J.; Warren, J. E.; Robertson, C. M.; Blanc, F.; Darling, G. R.; Berry, N. G., Guest-Adaptable and Water-Stable Peptide-Based Porous Materials by Imidazolate Side Chain Control. Angewandte Chemie 2014, 126 (1), 197-202) and PXRD pattern simulated from a molecular model of ZnCar coordination polymer (ZnCar-CP). ZnCar-MOF and ZnCar-CP structures are shown along the b crystallographic axes. Broadening of experimental spectrum relative to simulated spectrum is attributed to small size of individual crystallites composing the material as described in literature by Scherrer equation (see Patterson, A., The Scherrer formula for X-ray particle size determination. Physical review 1939, 56 (10), 978).

FIGS. 3A, FIG. 3B, FIG. 3C, and FIG. 3D demonstrate in vitro biocompatibility of ZIF-8 and ZnCar as measured by MTT assay.

FIG. 3A is a graph of cell viability of fibroblasts incubated with different concentrations of ZIF-8 and ZnCar for 24 h. Data is presented as average±standard deviation (n=6 to 12). % Viability is normalized to media only control.

FIG. 3B is a graph of cell viability of fibroblasts incubated with different concentrations of ZIF-8 and ZnCar for 48 h. Data is presented as average±standard deviation (n=6 to 12). % Viability is normalized to media only control.

FIG. 3C is a graph of cell viability of dendritic cells incubated with different concentrations of ZIF-8 and ZnCar for 24 h. Data is presented as average±standard deviation (n=6 to 12). % Viability is normalized to media only control.

FIG. 3D is a graph of cell viability of dendritic cells incubated with different concentrations of ZIF-8 and ZnCar for 48 h. Data is presented as average±standard deviation (n=6 to 12). % Viability is normalized to media only control.

FIG. 4A is a scanning electron micrograph image of ZnCar-CpG.

FIG. 4B includes a graph showing zeta potential of ZnCar after incubation in HEPES buffer or PBS buffer, and ZnCar-CpG after incubation in HEPES buffer (incubation time 3 h for all samples), and also includes a Table of zeta potentials of ZnCar after incubation in HEPES buffer, ZnCar after incubation in PBS buffer, and ZnCar-CpG after incubation in HEPES buffer. Reported as average±standard deviation (SD). Number of data points 10-20.

FIG. 4C is a graph showing cell viability of macrophages incubated with different concentrations of ZnCar (left) and ZnCar-CpG (right) for 24 h measured by MTT assay. Empty ZnCar did not contain CpG and contained equivalent concentration of ZnCar as ZnCar-CpG material at indicated concentrations. Data is presented as average±standard deviation (n=3). % Viability is normalized to media only control.

FIG. 4D is a graph showing nitric oxide (NO) released by macrophages with Soluble CpG (right), ZnCar (left), and ZnCar-CpG (middle). Data is presented as average±standard deviation (n=3).

FIG. 4E is a graph showing Secretion of TNF-α by macrophages incubated with indicated concentrations of Soluble CpG (right), ZnCar-CpG (middle), or ZnCar (left) for 24 hr. ZnCar did not contain CpG and contained equivalent concentration of ZnCar as ZnCar-CpG at indicated concentrations. Concentration of IL-6 and TNF-α were determined by ELISA. Data is presented as average±standard deviation (n=2 for IL-6 and n=3 for TNF-α). Unpaired t-tests were performed between ZnCar-CpG and Soluble CpG at each concentration. Significance reported as **=p<0.01, ***=p<0.001, ****=p<0.0001.

FIG. 4F is a graph showing secretion of IL-6 by macrophages incubated with indicated concentrations of Soluble CpG (right), ZnCar-CpG (middle), or ZnCar (left) for 24 hr. ZnCar did not contain CpG and contained equivalent concentration of ZnCar as ZnCar-CpG at indicated concentrations. Concentration of IL-6 and TNF-α were determined by ELISA. Data is presented as average±standard deviation (n=2 for IL-6 and n=3 for TNF-α). Unpaired t-tests were performed between ZnCar-CpG and Soluble CpG at each concentration. Significance reported as *=p<0.05, **=p<0.01.

FIG. 5A is a scanning electron micrograph image of OVA/ZnCar.

FIG. 5B is a graph showing OVA release from OVA/ZnCar after 24 hours at pH 7.4 and 5.0.

FIG. 5C is a graph showing cell viability of dendritic cells incubated with different concentrations of ZnCar (left), OVA/ZnCar (middle), and OVA/ZnCar-CpG (right) for 24 h measured by MTT assay. Blank ZnCar did not contain OVA and contained equivalent concentration of ZnCar as OVA/ZnCar material at indicated concentrations. Data is presented as average±standard deviation (n=3). % Viability is normalized to media only control.

FIG. 5D is a graph showing relative MHC I presentation by BMDCs for ZnCar (bottom, triangle), OVA/ZnCar (top, diamond), and OVA/ZnCar-CpG (middle, diamond). Data is presented as the average fold change vs. soluble protein±standard deviation (n=3).

FIG. 5E is a graph showing serum OVA-specific total IgG antibody titers of mice (n=5 per group) vaccinated intramuscularly on days 0 and 21 with indicated experimental groups. Groups receiving OVA received 10 μg per mouse per dose. Groups receiving CpG received 10 μg per mouse per dose. Data is presented as average±standard deviation.

FIG. 5F is a graph showing serum OVA-specific total IgG2c antibody titers of mice (n=5 per group) vaccinated intramuscularly on days 0 and 21 with indicated experimental groups. Groups receiving OVA received 10 μg per mouse per dose. Groups receiving CpG received 10 μg per mouse per dose. Data is presented as average±standard deviation.

FIG. 5G is a graph showing serum OVA-specific total IgG1 antibody titers of mice (n=5 per group) vaccinated intramuscularly on days 0 and 21 with indicated experimental groups. Groups receiving OVA received 10 μg per mouse per dose. Groups receiving CpG received 10 μg per mouse per dose. Data is presented as average±standard deviation.

FIG. 6A is a scanning electron micrograph image of ZnCar-HA.

FIG. 6B is a graph showing secretion of IL-6 and TNF-α incubated with indicated concentrations of HEPES, solHA+solCpG, and ZnCar-CpG+ZnCar-HA. Concentration of IL-6 and TNF-α were determined by ELISA. Data is presented as average±standard deviation (n=4 to 5).

FIG. 6C is a graph showing serum HA-specific total IgG antibody titers of mice (n=5 per group) vaccinated intramuscularly on days 0 and 21 with indicated experimental groups. Groups receiving HA received 10 μg per mouse per dose (HA type: COBRA P1). Groups receiving CpG received 10 μg per mouse per dose. Data is presented as average±standard deviation.

FIG. 6D is a graph showing serum HA-specific total IgG2c antibody titers of mice (n=5 per group) vaccinated intramuscularly on days 0 and 21 with indicated experimental groups. Groups receiving HA received 10 μg per mouse per dose (HA type: COBRA P1). Groups receiving CpG received 10 μg per mouse per dose. Data is presented as average±standard deviation.

FIG. 6E is a graph showing serum HA-specific total IgG1 antibody titers of mice (n=5 per group) vaccinated intramuscularly on days 0 and 21 with indicated experimental groups. Groups receiving HA received 10 μg per mouse per dose (HA type: COBRA P1). Groups receiving CpG received 10 μg per mouse per dose. Data is presented as average±standard deviation.

FIG. 7A is a scanning electron micrograph image of ZnCar.

FIG. 7B is a scanning electron micrograph image of a coordination polymer formed with Zinc and His-Asp-OH.

FIG. 7C is a scanning electron micrograph image of a coordination polymer formed with Zinc and His-Ala-OH.

FIG. 7D is a scanning electron micrograph image of a coordination polymer formed with Zinc and H-His-Leu-OH.

FIG. 7E is a scanning electron micrograph image of a coordination polymer formed with Zinc and H-His-Tyr-OH.

FIG. 7F is a scanning electron micrograph image of a coordination polymer formed with Zinc and H-Asp(His-OH)—OH.

FIG. 7G is a scanning electron micrograph image of a coordination polymer formed with Zinc and H-Gly-Gly-His-OH.

FIG. 7H is a scanning electron micrograph image of a coordination polymer formed with Zinc and H-Gly-His-Gly-OH.

FIG. 7I is a scanning electron micrograph image of a coordination polymer formed with manganese and H-Gly-Gly-His-OH.

FIG. 7J is a scanning electron micrograph image of a coordination polymer formed with manganese and H-Asp(His-OH)—OH.

FIG. 7K is a scanning electron micrograph image of a coordination polymer formed with manganese and H-Gly-His-Gly-OH.

FIG. 7L is a scanning electron micrograph image of a coordination polymer formed with copper and H-Gly-His-Lys-OH.

FIG. 7M is a scanning electron micrograph image of a coordination polymer formed with copper sulfate salt and histamine.

FIG. 7N is a scanning electron micrograph image of a coordination polymer formed with copper acetate salt and histamine.

FIG. 8 includes scanning electron micrograph images of ZnCar polycrystalline spheres grown in HEPES buffer pH=7.4 at room temperature and no stirring (time=4 days).

FIG. 9A includes scanning electron micrograph images of ZnCar polycrystalline spheres grown in HEPES buffer pH=7.4 at room temperature and no stirring, after the spheres were cut with a razor.

FIG. 9B includes comparison of scanning electron micrographs of ZnCar synthesized at room temperature and no stirring (left) vs. ZnCar synthesized at 37° C. Micrograph on the left is zoom on a flat surface of a hemisphere obtained by cutting original polycrystalline sphere in half.

FIG. 10 is an experimental PXRD pattern of ZnCar (as synthesized, HEPES buffer pH=7.4, 37° C., 18 h, stirring), compared with experimental PXRD pattern of ZnCar (as synthesized, HEPES buffer pH=7.4, room temperature, 4 days, no stirring).

FIG. 11 Experimental PXRD pattern of ZnCar (as synthesized, HEPES buffer pH=7.4, 37° C., 18 h, stirring), compared with PXRD pattern simulated from crystal structure of zinc-carnosine material (as synthesized, HEPES buffer pH=7.4+ethanol, room temperature, no stirring) and PXRD pattern simulated from crystal structure of ZnCar-MOF (CCDC 949242) reported in literature. See Katsoulidis, A. P.; Park, K. S.; Antypov, D.; Marti-Gastaldo, C.; Miller, G. J.; Warren, J. E.; Robertson, C. M.; Blanc, F.; Darling, G. R.; Berry, N. G., Guest-Adaptable and Water-Stable Peptide-Based Porous Materials by Imidazolate Side Chain Control. Angewandte Chemie 2014, 126 (1), 197-202.

FIG. 12A is a scanning electron micrograph image of ZnCar-MOF (DMF solvate) formed by synthesis according to Katsoulidis et al. Reaction mixture was stirred during synthesis.

FIG. 12B is an experimental PXRD spectrum of ZnCar-MOF (DMF solvate) formed by synthesis according to Katsoulidis et al. Reaction mixture was stirred during synthesis. Experimental PXRD overlaid with PXRD simulated from crystal structure of ZnCar-MOF (DMF solvate; CCDC 949241).

FIG. 13 includes experimental PXRD pattern of ZnCar-MOF (as synthesized, unstirred during synthesis), compared with experimental PXRD pattern of ZnCar-MOF (as synthesized, stirred during synthesis) and PXRD pattern simulated from crystal structure of ZnCar-MOF reported in Katsoulidis et al. Due to experimental conditions, ZnCar-MOF is in a form of DMF solvate (CCDC 949241).

FIG. 14A is an experimental PXRD pattern of ZnCar (as synthesized in HEPES buffer, stirred during synthesis), compared with experimental PXRD pattern of ZnCar-MOF DMF solvate (as synthesized, unstirred during synthesis) and PXRD pattern simulated from crystal structure of ZnCar-MOF DMF solvate reported in Katsoulidis et al.

FIG. 14B is a scanning electron micrograph of ZnCar-MOF DMF solvate (as synthesized, unstirred during synthesis).

FIG. 15A is a scanning electron micrograph of ZnCar after incubation for 3 h in HEPES at pH=7.4.

FIG. 15B is a scanning electron micrograph of ZnCar after incubation for 3 h in PBS buffer at pH=7.4.

FIG. 16A is a scanning electron micrograph of ZnCar formed at 60× scale up.

FIG. 16B is a PXRD spectrum of ZnCar formed at 60× scale up, with PXRD overlaid with that of ZnCar formed at smaller scale.

FIG. 17A is a graph showing viability of fibroblasts incubated with different concentrations of zinc acetate dihydrate and zinc nitrate hexahydrate measured by MTT assay. Data is presented as average±standard deviation (n=3 to 6). % Viability is normalized to media only control.

FIG. 17B is a graph showing cell viability of fibroblasts incubated with different concentrations of L-carnosine and 2-methylimidazole measured by MTT assay. Data is presented as average standard deviation (n=3 to 6). % Viability is normalized to media only control.

FIG. 18 is a graph showing a release profile of OVA from OVA/ZnCar in 0.1M HEPES buffer pH=7.4 at time points ranging from 0 to 24 h.

FIG. 19A is a graph showing IgG titer for mice vaccinated with different compositions. BALB/cJ mice (n=18) were vaccinated on a prime+boost schedule (day 0 and day 21). Sera was collected on days 14, 28, and 42 for the quantification of anti-Y2 antibodies via ELISA. Y2 is a designer influenza hemagglutinin protein.

FIG. 19B is a graph showing IgG1 titer for mice vaccinated with different compositions. BALB/cJ mice (n=18) were vaccinated on a prime+boost schedule (day 0 and day 21). Sera was collected on days 14, 28, and 42 for the quantification of anti-Y2 antibodies via ELISA.

FIG. 19C is a graph showing IgG2a titer for mice vaccinated with different compositions. BALB/cJ mice (n=18) were vaccinated on a prime+boost schedule (day 0 and day 21). Sera was collected on days 14, 28, and 42 for the quantification of anti-Y2 antibodies via ELISA.

FIG. 20 is a graph showing concentration of compositions in lung (PFU/100 mg). *P-value<0.05.

FIG. 21 is a graph showing TNF-α concentration of compositions with different CpG concentrations according to some embodiments described herein. Here the composition is at ideal storage −20° C. or 40° C. for three months before evaluation.

FIGS. 22A and 22B are graphs showing results of antibody stability assays used to determine whether the structure of Y2 was intact after three months of storage at two different temperatures. Significance reported as ***=p<0.001, ****=p<0.0001.

FIG. 23 is a graph showing Abs_{400 nm} over time for some embodiments described herein.

FIG. 24A is a graph of IgG endpoint titers when the excipient mannan is added to the final composition as in some embodiments described herein.

FIG. 24B is a graph of IgG1 endpoint titers when the excipient mannan is added to the final composition as in some embodiments described herein.

FIG. 24C is a graph of IgG2c endpoint titers when the excipient mannan is added to the final composition as in some embodiments described herein.

FIG. 25A shows DOX/ZnCar (encapsulation method). Theoretical mass % of DOX loaded in the material (from left to right): 0.2%, 1%, 5%, 10% before workup.

FIG. 25B shows DOX/ZnCar (encapsulation method). Theoretical mass % of DOX loaded in the material (from left to right): 0.2%, 1%, 5%, 10% after lyophilization.

FIG. 26A shows ZnCar-DOX (surface adsorption method). Theoretical mass % of DOX loaded in the material (from left to right): 5%, 10% before workup.

FIG. 26B shows ZnCar-DOX (surface adsorption method). Theoretical mass % of DOX loaded in the material (from left to right): 5%, 10% after the first spin in a microcentrifuge.

FIG. 26C shows ZnCar-DOX (surface adsorption method). Theoretical mass % of DOX loaded in the material (from left to right): 5%, 10% after lyophilization.

FIG. 27 shows DOX release from DOX/ZnCar material after incubation in 0.1 M HEPES buffer (pH=7.4) and 0.3 M acetate buffer (pH=5.0) for 1 h at 37° C.

### DETAILED DESCRIPTION

Embodiments described herein can be understood more readily by reference to the following detailed description and examples and their previous and following descriptions. Elements, apparatus, and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less (e.g., 1.0 to 5.3, or 4.7 to 10.0, or 3.6 to 7.9).

All ranges disclosed herein are also to be considered to include the end points of the range, unless expressly stated otherwise. For example, a range of “between 5 and 10” or “5 to 10” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

### Example 1—ID Zn-Carnosine Coordination Polymer for Antigen Delivery

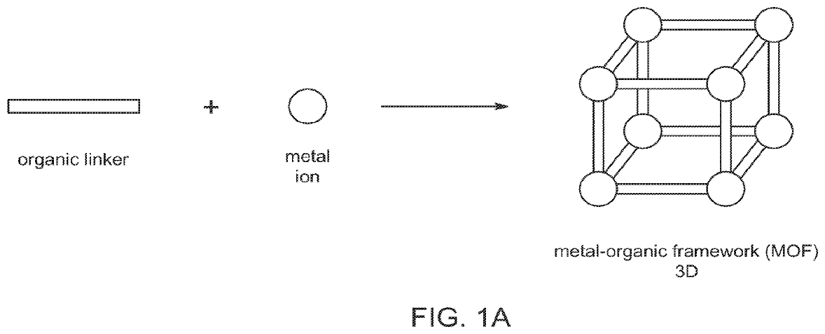

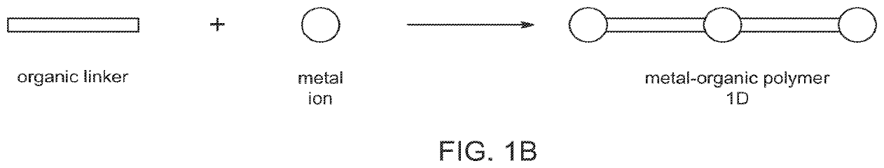

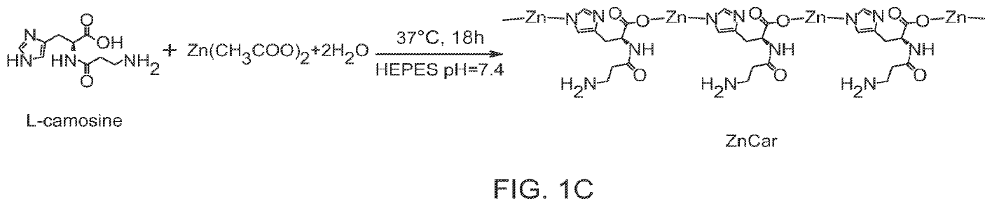

Metal-based coordination polymers with organic ligands have been reported as 1-dimensional, 2D and 3D structures, with 2D and 3D structures containing internal pores and referred to as metal-organic frameworks (MOFs) (FIG. 1A and FIG. 1B). Here, the use of a coordination polymer comprised of the dipeptide carnosine and zinc (Zn²⁺) (ZnCar) for incorporation of protein and delivery of adjuvant as an influenza vaccine is disclosed. Carnosine is a naturally occurring peptide in muscle and brain tissue, whose histidine has an imidazole group imparting acid sensitivity that will break its coordination with metal ions near a pH of 5.0. This pH switch allows for a triggered release upon internalization by phagocytic cells (e.g., macrophages, dendritic cells (DCs)) and exposure to the acidic pH of the endo/lysosome. This is ideal for a vaccine carrier.

A 3D MOF constructed from carnosine and Zn²⁺ (ZnCar-MOF) has been reported previously. See Katsoulidis, A. P.; Park, K. S.; Antypov, D.; Marti-Gastaldo, C.; Miller, G. J.; Warren, J. E.; Robertson, C. M.; Blanc, F.; Darling, G. R.; Berry, N. G., Guest-Adaptable and Water-Stable Peptide-Based Porous Materials by Imidazolate Side Chain Control. Angewandte Chemie 2014, 126 (1), 197-202; and Pullin, T. M. Biomolecule encapsulation in biocompatible metal-organic frameworks. 2019. However, the fabrication process for this MOF relies on harsh solvents (e.g., dimethylformamide, ethanol) and elevated temperature, which can denature protein antigens and lead to the generation of non-neutralizing antibodies. Generation of metal-based coordination polymers in more biologically relevant solutions could result in enhanced vaccine efficacy, compared to those in organic solutions, however, different constructs may form in place of 3D MOFs. One such construct is a 1D infinite coordination polymer. 1D polymers are formed by metal ions interconnected by bridging linkers with two extension points per linker (FIG. 1B), while other coordination sites of the metal ions are capped with ligands that lack extension points, such as most solvent molecules. While MOFs are typically more porous structures than 1D polymers, both of these types of coordination materials are capable of serving as drug delivery vehicles via drug attachment to the surface or encapsulation in the void space within the structures.

Influenza vaccines are used to prevent pandemics like those observed in 1918, 1957, 1968, and 2009, and predicted to occur in the future with a H3N2 strain. The 2009 H1N1 influenza pandemic strain led to over a half-million deaths worldwide. Historically, the strains selected for seasonal influenza vaccines can differ from circulating strains because of antigenic shift and/or drift, which can result in significantly reduced vaccine efficacy, therefore more broadly or universal approaches are needed to provide enhanced protection over the current vaccines. To identify a broadly reactive influenza antigen that can elicit protective responses against a wider array of circulating influenza viruses, Computationally Optimized Broadly Reactive Antigen (COBRA) was developed. COBRA uses iterative layered consensus building from hemagglutinin (HA) sequences of circulating influenza isolates to construct an antigen capable of eliciting broadly reactive immune response. These antigens could protect against past and future seasonal and novel pandemic influenza strains including pandemic H1N1 subtypes.

A broadly protective and safely applied COBRA HA vaccine has many advantages compared to conventional seasonal flu vaccine formulations; however, recombinant protein antigens tend to activate the immune response only weakly without the addition of an adjuvant. For this reason, toll-like receptor 9 (TLR 9) agonist CpG with COBRA H1 HA has been formulated. CpG is FDA approved as an adjuvant in hepatitis B vaccine Heplisav-BK. The evaluation of a ZnCar metal-based coordination polymer for delivery of COBRA H1 HA and CpG for application as an influenza vaccine is described hereinbelow. The platform was characterized by scanning electron microscopy (SEM), high performance liquid chromatography (HPLC), zeta potential, and powder X-ray diffraction (PXRD) spectroscopy, and generated x-ray diffraction spectra were compared to the previously reported ZnCar-MOF. The humoral and cellular response was evaluated with model antigen ovalbumin (OVA) and then activity was characterized with COBRA H1 HA in a mouse model. Overall, a new metal-organic platform that can be applied for generation of subunit vaccines is disclosed.

### Materials and Methods

All chemicals were purchased form Sigma (St. Louis, MO) and used as purchased, unless otherwise indicated. Assays, biologics, and disposables were purchased from Thermo Fisher Scientific (Waltham, MA) unless otherwise indicated.

### Synthesis of Empty and Loaded ZnCar

0.1M HEPES solution was prepared by diluting an aliquot of sterile 1M HEPES buffer solution of pH 7.4 (Corning, Corning, NY) in molecular biology grade water (Corning) and adjusting the pH of the resulting solution to 7.4 with 1M NaOH aqueous solution. Carnosine (0.227 mmol) was dissolved in 5 ml of HEPES solution. Zn(CH₃COO)₂·2H₂O (0.227 mmol) was dissolved in 10 ml of HEPES solution. Carnosine and Zn(CH₃COO)₂·2H₂O solutions were mixed together, and the reaction mixture was stirred at 37° C. for 18 h. The reaction mixture was cooled down to room temperature and the obtained precipitate was isolated by centrifugation (22,000×g, 20 min, 4° C.). The pellet was washed with Milli-Q water (twice), re-suspended in 5 mL of Milli-Q water, frozen at −80° C. for 10 min and lyophilized to produce a zinc-carnosine coordination material (ZnCar) as a white powder (52 mg).

OVA/ZnCar was synthesized by mixing carnosine (51.3 mg, 0.227 mmol), Zn(CH₃COO)₂·2H₂O (50 mg, 0.227 mmol), and ovalbumin (OVA) (4 mg, 0.000093 mmol; Endofit OVA) in 15 ml of HEPES buffer (0.1M, pH=7.4). The reaction mixture was stirred at 37° C. for 18 h. The reaction mixture was cooled down to room temperature and the obtained precipitate was isolated by centrifugation (22,000×g, 20 min, 4° C.). Supernatant (contained unencapsulated OVA) was removed, and the pellet was washed with Milli-Q water (twice), re-suspended in 5 mL of Milli-Q water, frozen at −80° C. for 10 min and lyophilized to produce OVA-loaded zinc-carnosine coordination material (OVA/ZnCar) as a white powder. To characterize OVA loading in OVA/ZnCar material, samples were decomposed in acetate buffer (pH=5.0) at 1 mg/mL concentration, carnosine removed via a 3 kDa Amicon Ultra-0.5 centrifugal filter, and the retentate analyzed with a BCA assay. OVA loading was 4.28 μg/mg, corresponding to an encapsulation efficiency of 21.4%.

CpG ODN 1826 (CpG) (Invivogen, San Diego, CA) was adsorbed on ZnCar by mixing a suspension of ZnCar in HEPES buffer (0.1M, pH=7.4) and an aliquot of CpG stock solution in sterile water (40 μM), resulting in the formation of ZnCar-CpG material. Loading of CpG in ZnCar-CpG was confirmed by measuring absorbance of the supernatant (260 nm) after centrifugation (22,000×g, 20 min, 4° C.). Loading and encapsulation efficiency were determined to be 52.9 μg/mg and 100%, respectively. OVA/ZnCar-CpG material was synthesized in the identical way, with OVA/ZnCar used in place of blank ZnCar.

COBRA P1 HA (referred to as HA; containing a His-tag) was used and generated as previously indicated. (Carter D M et al. Design and Characterization of a Computationally Optimized Broadly Reactive Hemagglutinin Vaccine for H1N1 Influenza Viruses. J Virol 2016, 90 (9), 4720-4734; Allen J D et al. Split Inactivated Cobra Vaccine Elicits Protective Antibodies against H1N1 and H3N2 Influenza Viruses. PLoS One 2018, 13 (9), e0204284; Darricarrère N et al. Development of a Pan-H1 Influenza Vaccine. J Virol 2018, 92 (22), e01349-18; Sautto G A. Computationally Optimized Broadly Reactive Antigen Subtype-Specific Influenza Vaccine Strategy Elicits Unique Potent Broadly Neutralizing Antibodies against Hemagglutinin. J Immunol 2020, 204 (2), 375-385.) HA was loaded onto ZnCar by mixing a suspension of ZnCar in HEPES buffer (0.1M, pH=7.4) and an aliquot of HA in HEPES buffer, resulting in formation of ZnCar-HA material. HA loading in ZnCar-HA was evaluated with a BCA assay and determined to be 45.6 μg/mg.

### Synthesis of ZnCar-MOF and ZIF-8

ZnCar-MOF (see Katsoulidis, A. P.; Park, K. S.; Antypov, D.; Marti-Gastaldo, C.; Miller, G. J.; Warren, J. E.; Robertson, C. M.; Blanc, F.; Darling, G. R.; Berry, N. G., Guest-Adaptable and Water-Stable Peptide-Based Porous Materials by Imidazolate Side Chain Control. Angewandte Chemie 2014, 126 (1), 197-202) and ZIF-8 MOF (see Liang, K.; Ricco, R.; Doherty, C. M.; Styles, M. J.; Bell, S.; Kirby, N.; Mudie, S.; Haylock, D.; Hill, A. J.; Doonan, C. J., Biomimetic mineralization of metal-organic frameworks as protective coatings for biomacromolecules. Nature communications 2015, 6 (1), 1-8) were synthesized as previously indicated.

### Formation of ZnCar and Other Metal and Organic Coordination Polymers

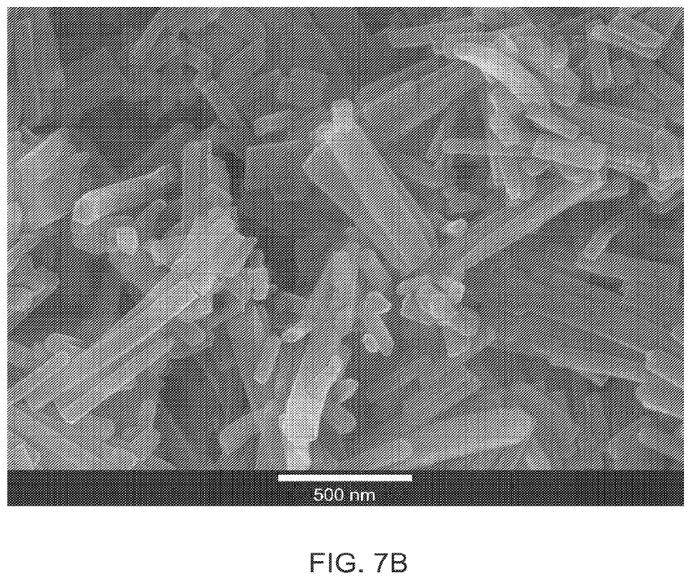

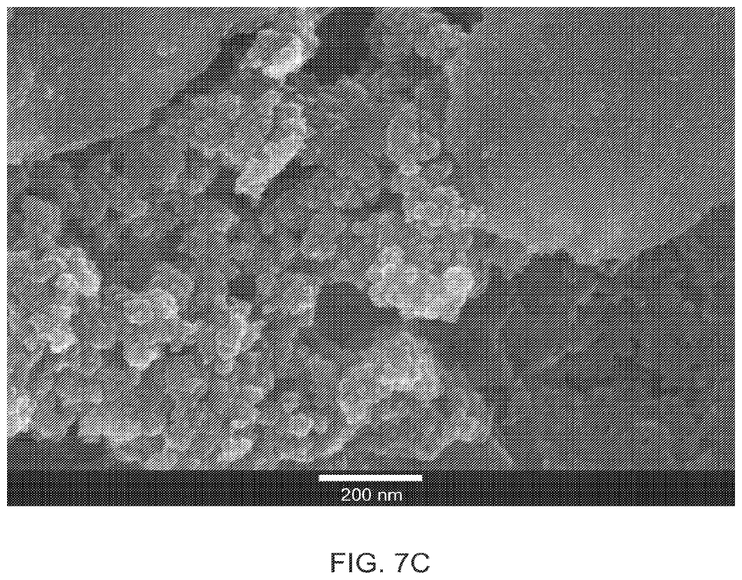

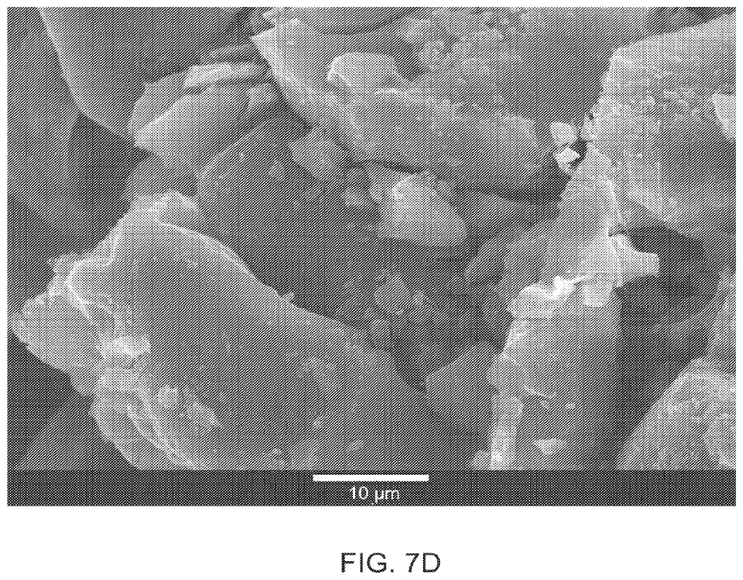

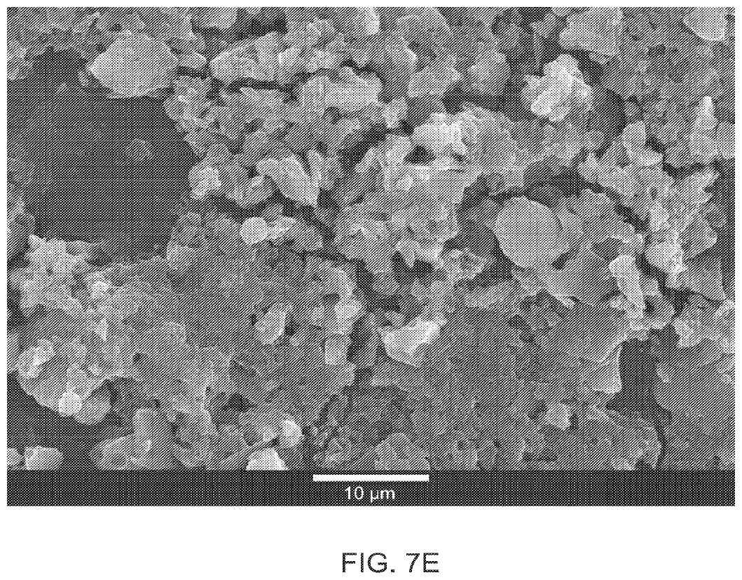

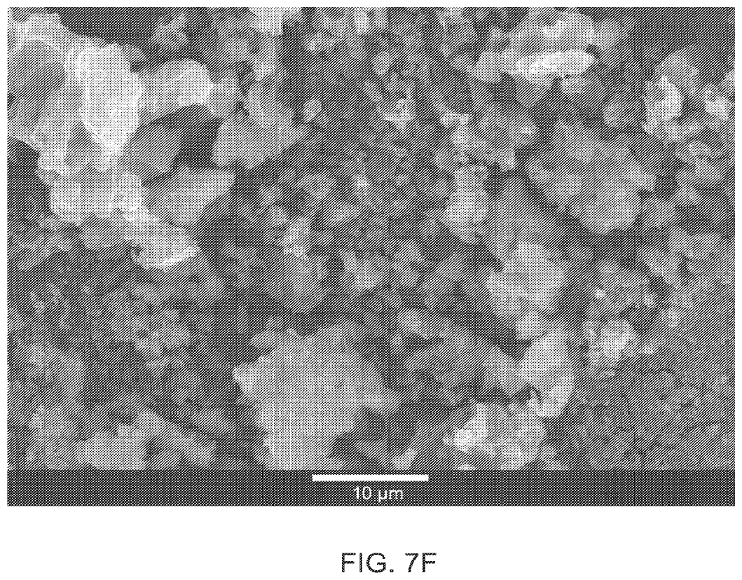

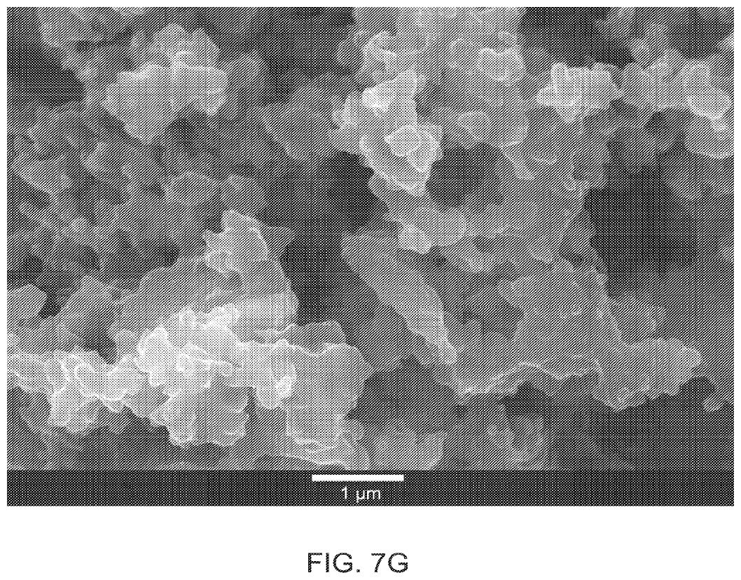

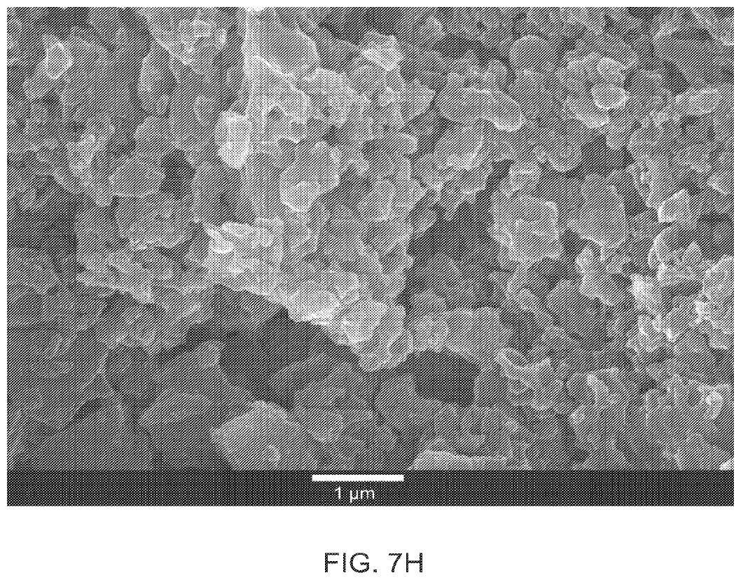

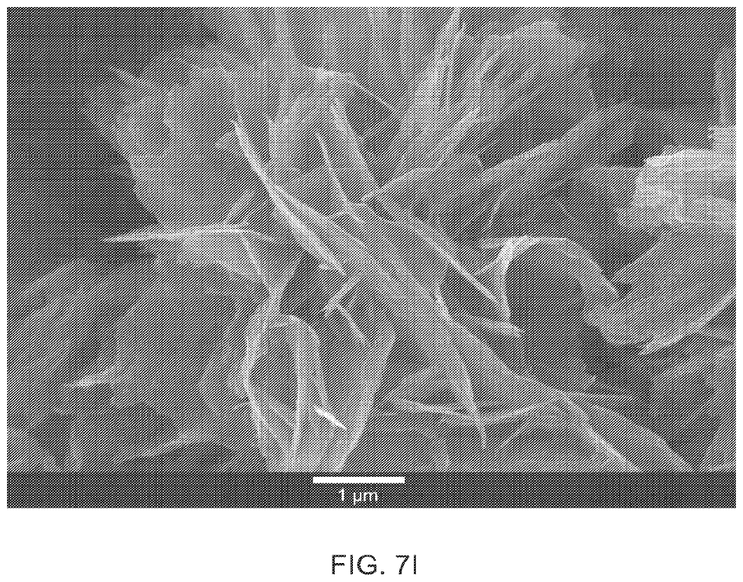

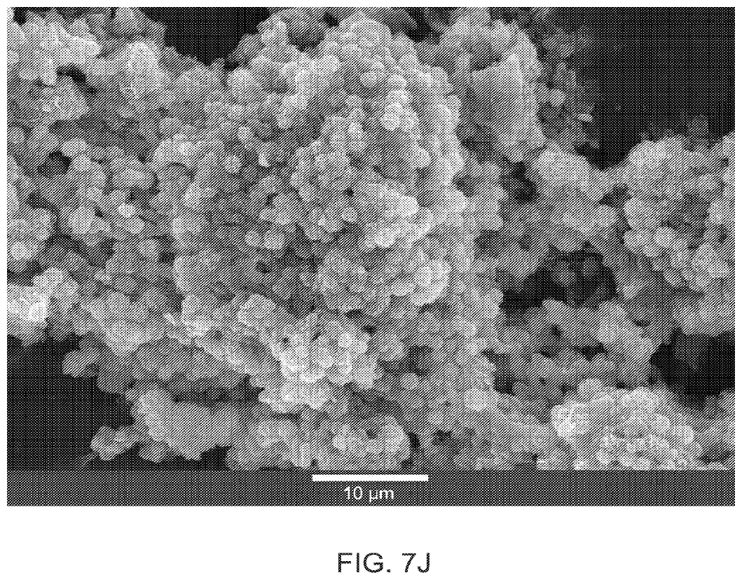

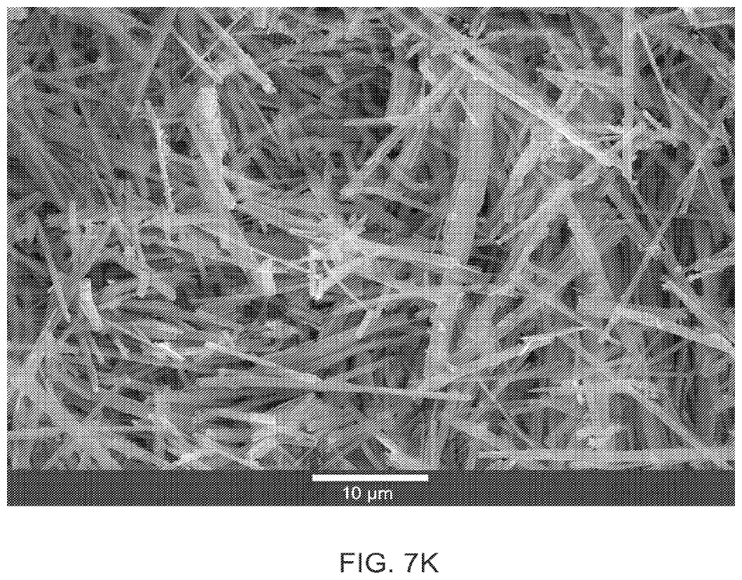

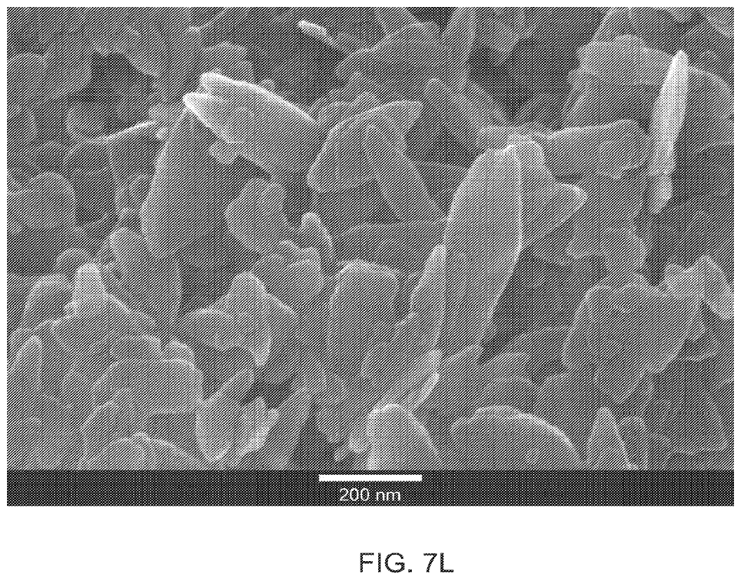

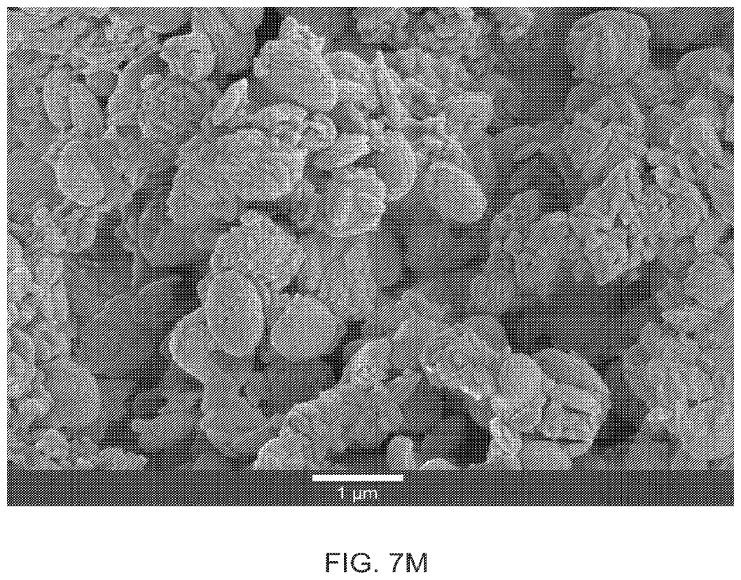

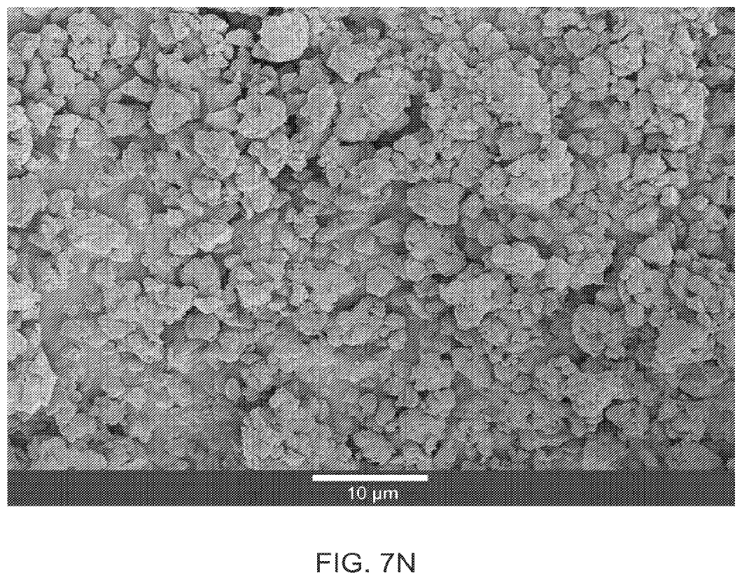

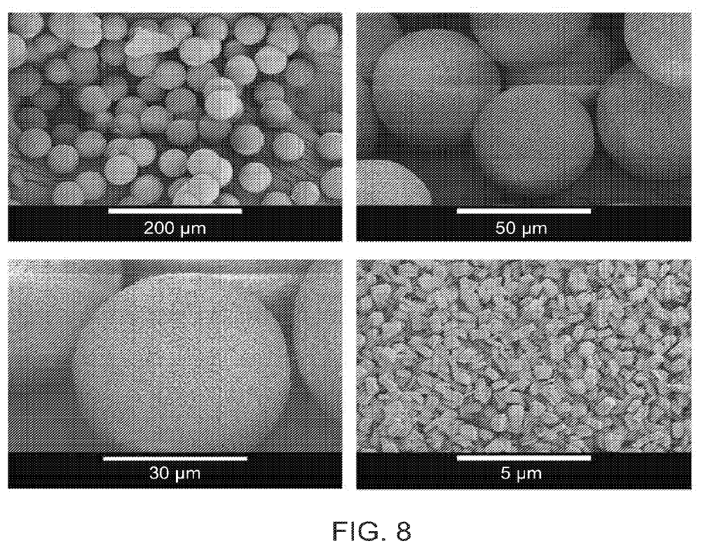

Coordination polymers can be formed with metals and inorganic peptides. Formation of the ZnCar composition is given in the micrograph in FIG. 7A. Additionally, other coordination polymers with zinc and other peptides have been formed. These peptides include FIG. 7B His-Asp-OH; FIG. 7C His-Ala-OH; FIG. 7D H-His-Leu-OH; FIG. 7E H-His-Tyr-OH; FIG. 7F H-Asp(His-OH)—OH; FIG. 7G H-Gly-Gly-His-OH; and FIG. 7H H-Gly-His-Gly-OH. In addition to zinc, coordination polymers have been formed with manganese and peptides. These peptides include: FIG. 7I H-Gly-Gly-His-OH; FIG. 7J H-Asp(His-OH)—OH; and FIG. 7K H-Gly-His-Gly-OH. Also, coordination polymers have been formed with copper and H-Gly-His-Lys-OH (FIG. 7L). Additionally, two copper salts were used to form two coordination polymers with histamine, including the sulfate salt (FIG. 7M) and acetate salt (FIG. 7N).

### Characterization

### ZnCar Structure Determination

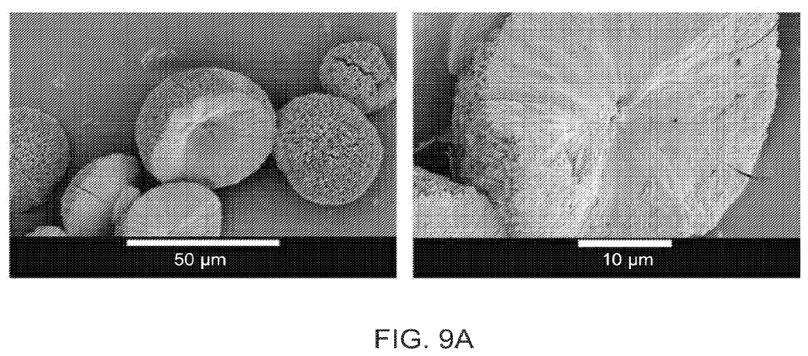

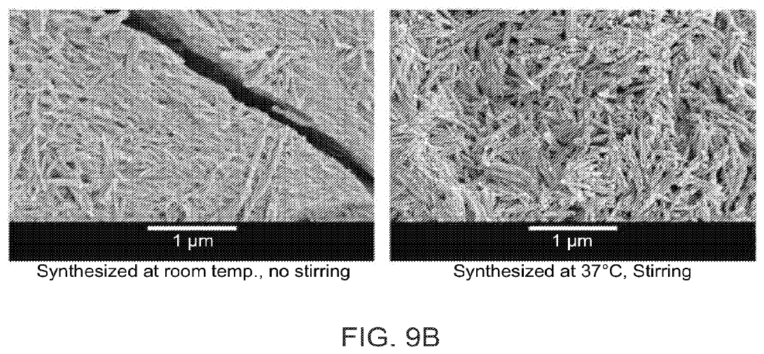

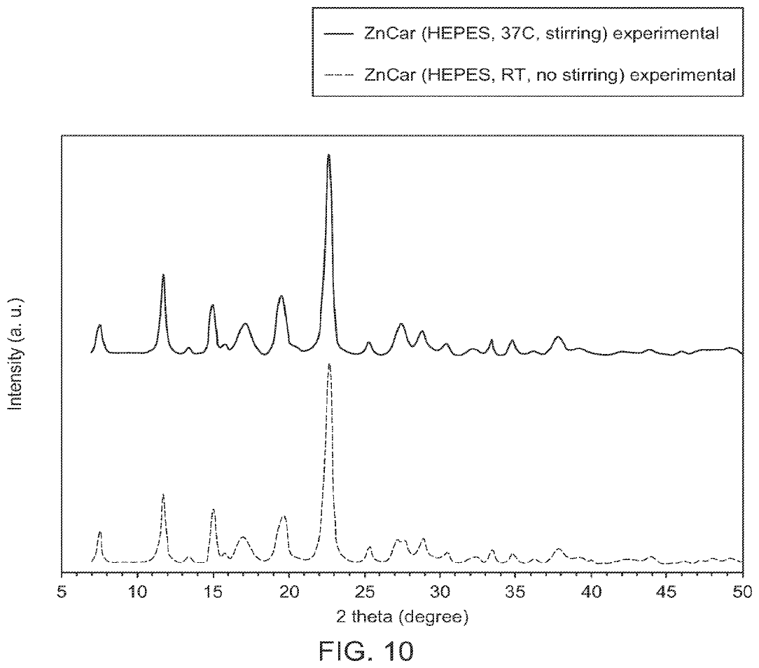

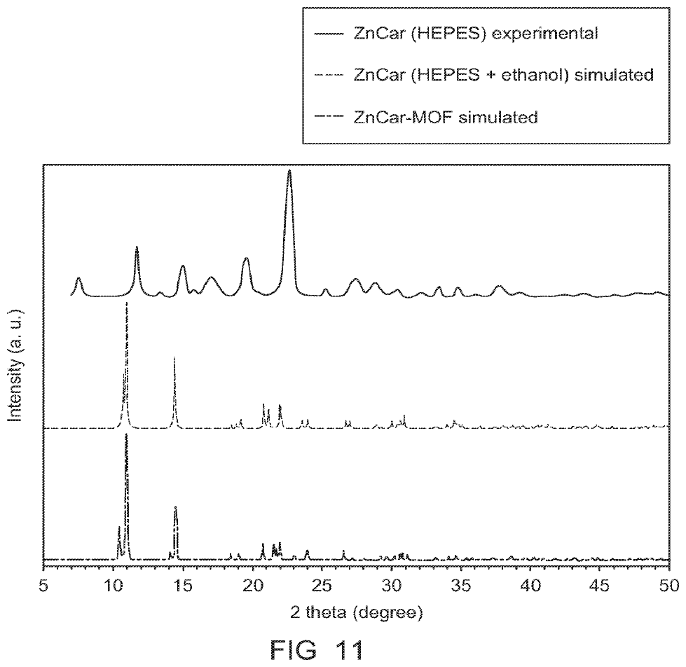

Numerous attempts to obtain a crystal of ZnCar material suitable for crystal structure determination were performed but none of the methods produced a crystal of suitable quality. Regular synthesis conditions (HEPES buffer pH=7.4, 37° C., 18 h, stirring) produced nanofibrous material (FIG. 7a) not suitable for single X-ray diffraction (SXRD) analysis due to the small crystal dimensions. Performing the synthesis without stirring (HEPES buffer pH=7.4, 37° C., 18 h, no stirring) did not improve crystallinity of the product. When crystals were grown at room temperature with no stirring, spherical aggregates composed of microcrystals were obtained (FIG. 8); similar results were obtained when crystals were grown at 4° C. with no stirring. When these polycrystalline spheres were cut in halves with a razor, SEM demonstrated that the spheres interior consists of fibers very similar to the fibers synthesized at 37° C. (FIG. 9A and FIG. 9B). Experimental PXRD of ZnCar synthesized at room temperature fully matched experimental PXRD of ZnCar synthesized at 37° C. (FIG. 10), indicating that it is the same compound. Unfortunately, spherical aggregates obtained by room temperature synthesis (25-50 m) were not suitable for SXRD due to their polycrystallinity, and microcrystals composing those spheres (1 m or less) were not suitable for SXRD due to their small size and their intergrowth.

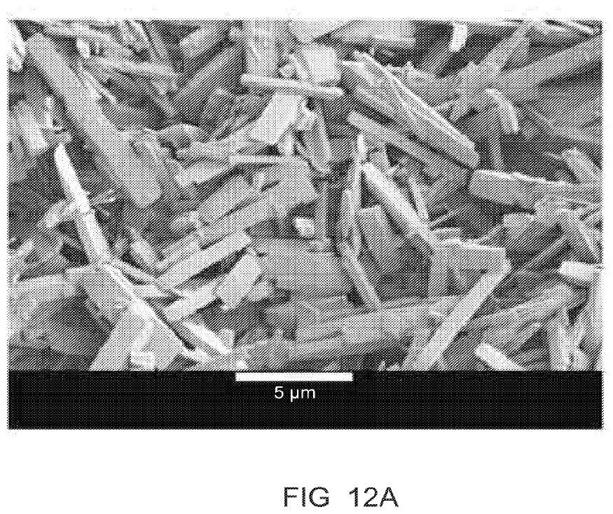

When synthesis conditions were altered to include ethanol (HEPES buffer pH=7.4, ethanol, no stirring; aqueous solvent:ethanol at 2:1 ratio), SXRD-suitable crystalline material was formed. When this crystalline material was analyzed with SXRD, it revealed crystal structure of 3D ZnCar-MOF reported in literature. See, Katsoulidis, A. P.; Park, K. S.; Antypov, D.; Marti-Gastaldo, C.; Miller, G. J.; Warren, J. E.; Robertson, C. M.; Blanc, F.; Darling, G. R.; Berry, N. G., Guest-Adaptable and Water-Stable Peptide-Based Porous Materials by Imidazolate Side Chain Control. Angewandte Chemie 2014, 126 (1), 197-202. PXRD spectrum simulated from this crystal structure fully matched simulated PXRD spectrum of ZnCar-MOF but differed from experimental PXRD of ZnCar synthesized in just HEPES buffer (FIG. 11), indicating that those two materials are not the same. So, while addition of organic solvent like ethanol improved the crystallinity of the material, it resulted in the formation of a different structure; meanwhile, crystallinity of the material formed in the absence of ethanol was not suitable for SXRD.

Next, structure determination was attempted using microcrystal electron diffraction (microED) technique. However, even with cryogenic sample preparation diffraction from the ZnCar material was too weak to be used for the structure solution.

Due to the lack of SXRD-suitable or micro-ED-suitable material, molecular modeling was utilized. A molecular model of ZnCar structure was based on experimental PXRD spectrum of ZnCar (HEPES buffer pH=7.4, 37° C., 18 h, stirring) and was generated via Expo2014 software. See, Altomare, A.; Cuocci, C.; Giacovazzo, C.; Moliterni, A.; Rizzi, R.; Corriero, N.; Falcicchio, A., EXPO2013: a kit of tools for phasing crystal structures from powder data. Journal of Applied Crystallography 2013, 46 (4), 1231-1235. Crystal structure of ZnCar-MOF (CCDC 949242) was used as a starting point for the modeling.

### Endotoxin, Imaging, Zeta Potential

Endotoxin was evaluated using the Pierce LAL chromogenic endotoxin quantitation kit in accordance with the manufacturer instructions. All samples had undetectable levels of endotoxin (<0.1 EU/mg). SEM (Hitachi S-4700 with EDS, Tokyo, Japan) and PXRD (Rigaku SmartLab diffractometer, Tokyo, Japan) was carried out at UNC CHANL. For Cryo-EM imaging, ZnCar samples were suspended at 1 mg/mL in molecular grade water immediately prior to application to plasma-cleaned R1.2/1.3 Quantifoil Cu grids. Samples were blotted and frozen using a Vitrobot Mark IV. Data was collected at the UNC at Chapel Hill CryoEM Core Facility with a 200 keV Thermo Fisher Scientific Talos Arctica G3 equipped with a Gatan K3 direct electron detector. Zeta potential was determined on a NanoBrook 90Plus Zeta Particle Size Analyzer (Holtsville, NY).

### HPLC Analysis of Carnosine Content

To quantify ZnCar carnosine content, ZnCar was dissolved in a 0.1% trifluoroacetic acid (TFA). Carnosine loading was quantified by high performance liquid chromatography (HPLC, Agilent 1100 series, Santa Clara, CA) using a 0.1% TFA in water/0.1% TFA in acetonitrile gradient method through an Aquasil C18 column (150 mm length, 4.6 mm inner diameter, 5 m pore size) with a C8 guard column cartridge and a UV detection wavelength of 220 nm. Theoretical mass loading of carnosine at a 1:1 molar ratio was determined to be 77.37% w/w.

### In Vitro Experiments

All cell lines (RAW 264.7 murine macrophages, DC2.4 dendritic cells, and 3T3 fibroblasts; ATCC, Manassas, VA) were maintained according to ATCC guidelines. B3Z T cells were obtained from Dr. Nilabh Shastri (Johns Hopkins) and maintained and used as previously outlined. See, Broaders, K. E.; Cohen, J. A.; Beaudette, T. T.; Bachelder, E. M.; Frechet, J. M., Acetalated dextran is a chemically and biologically tunable material for particulate immunotherapy. Proc Natl Acad Sci USA 2009, 106 (14), 5497-502. Cell viability was determined with an MTT assay, as previously described. See, Chen, N.; Collier, M. A.; Gallovic, M. D.; Collins, G. C.; Sanchez, C. C.; Fernandes, E. Q.; Bachelder, E. M.; Ainslie, K. M., Degradation of acetalated dextran can be broadly tuned based on cyclic acetal coverage and molecular weight. Int J Pharm 2016, 512 (1), 147-157.

### Nitric Oxide and Proinflammatory Cytokine Secretion in Macrophages

Macrophages and fibroblasts were plated in a 96-well plate at a density of 5×10⁴ cells/well, incubated at 37° C. and left overnight to adhere. The cells were treated with complete medium, soluble CpG, ZnCar and ZnCar-CpG particles for 24 h. The isolated macrophage supernatants were collected and analyzed for nitrite concentration using the Griess reagent (Promega, Madison, WI) and IL-6 and TNF-α concentration using mouse TNF-α and IL-6 Ready-SET-Go sandwich ELISA kits.

### In Vivo Experiments

Mice (C57Bl/6) were purchased from The Jackson Laboratory (Bar Harbor, ME). All animal experiments were performed in accordance with UNC Institutional Animal Care and Use Committee (IACUC) approval. For experiments, mice received 10 μg of protein (OVA or COBRA HA) and 10 μg of CpG per a mouse per dose. Mice were vaccinated on day 0 and 21, and submandibular blood samples were taken on days 14, 28, and 42. On day 42, mice were sacrificed, and their spleens removed and processed for antigen recall. See Chen, N.; Gallovic, M. D.; Tiet, P.; Ting, J. P.; Ainslie, K. M.; Bachelder, E. M., Investigation of tunable acetalated dextran microparticle platform to optimize M2e-based influenza vaccine efficacy. J Control Release 2018, 289, 114-124.

### Antibody Titer Determination

Flat-bottomed high-binding polystyrene plates (Corning 29442-322) were coated overnight at 4° C. with 1 μg/mL COBRA Y2 HA in PBS. Plates were washed three times with 0.05% Tween 20 in PBS (PBST), then blocked for two hours at room temperature with 200 μL blocking buffer (3% w/v nonfat instant milk in PBS). Plates were washed three times again. Serum samples were diluted in 100 μL blocking buffer and added to the blocked plates for one hour. Plates were washed three times again. The appropriate secondary antibodies (Goat Anti-Mouse IgG Fc-HRP 1033-05, Goat Anti-Mouse IgG2c-HRP 1078-05, or Goat Anti-Mouse IgG1-HRP 1071-05, Southern Biotech) were diluted in blocking buffer to the highest dilution recommended by the manufacturer, and 100 L was added to each well for two hours at room temperature. Plates were washed five times with PBST and developed with tetramethylbenzidine (TMB) one component substrate (Southern Biotech 0410-01) before quenching with 2 N sulfuric acid. Development times were based on day 42 sera and all days of sera were developed for the same amount of time per each secondary antibody to allow comparison of titers between days. Plates were read for absorbance at 450 nm and corrected for background by subtracting absorbance at 570 nm. Antibody titers were determined by fitting a curve to the background-corrected absorbance vs. dilution using the “log(inhibitor) vs. response—Variable slope (four parameters)” model in Graphpad Prism 8, then interpolating the dilution value at which the curve intercepts the endpoint value as defined by Frey et al. using a 99.9% confidence level and twelve background controls. See, Frey, A.; Di Canzio, J.; Zurakowski, D. J. J. o. i. m., A statistically defined endpoint titer determination method for immunoassays. 1998, 221 (1-2), 35-41.

### Results

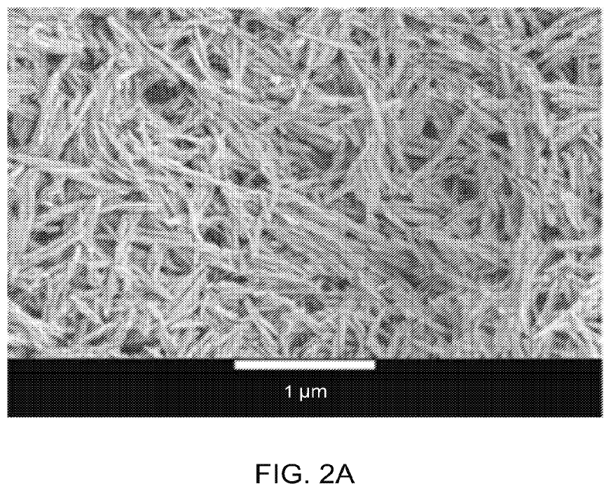

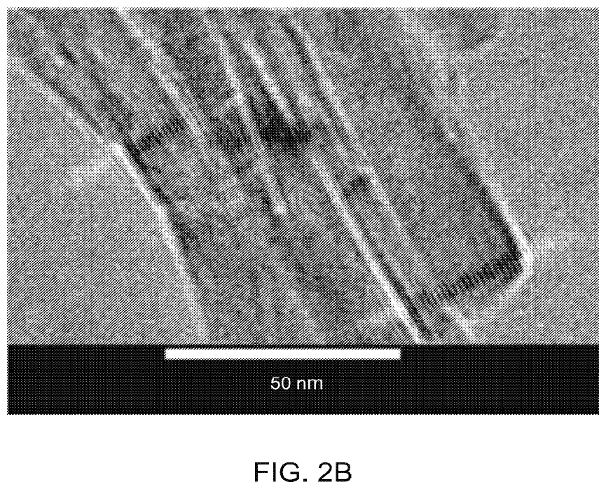

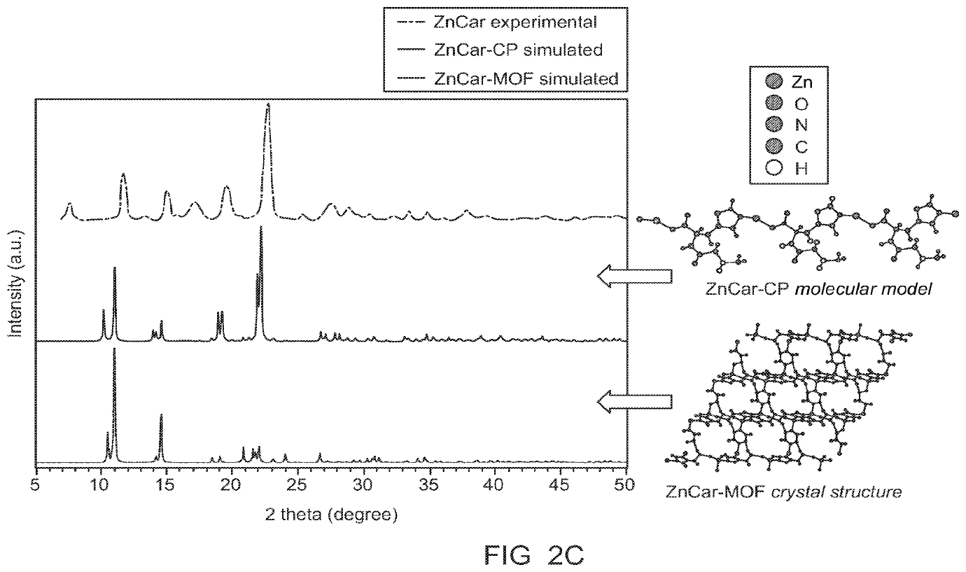

Zinc and carnosine were reacted in 0.1M HEPES at neutral pH with overnight stirring at 37° C. (FIG. 1C and FIG. 2A). HEPES buffer was chosen because it does not complex to zinc. The product (ZnCar) was determined to be composed of 77.00±2.59% carnosine by mass, consistent with a 1:1 molar ratio of zinc and carnosine. The PXRD spectrum of ZnCar was collected and compared to a simulated spectrum of ZnCar-MOF reported in literature (see Katsoulidis, A. P.; Park, K. S.; Antypov, D.; Marti-Gastaldo, C.; Miller, G. J.; Warren, J. E.; Robertson, C. M.; Blanc, F.; Darling, G. R.; Berry, N. G., Guest-Adaptable and Water-Stable Peptide-Based Porous Materials by Imidazolate Side Chain Control. Angewandte Chemie 2014, 126 (1), 197-202) and to a simulated spectrum of a 1D Zn-Carnosine coordination polymer (ZnCar-CP) (FIG. 2C), the structure of which was modeled utilizing Expo2014 software. The experimental PXRD pattern of ZnCar (the product generated in HEPES buffer) closely resembles the simulated powder diffraction pattern for a ZnCar-CP but differs from that of the ZnCar-MOF (FIG. 2C). Generation of a 1D Zn-Carnosine coordination polymer formed under the synthetic conditions was also supported by cryogenic electron microscopy (cryo-EM), which revealed lattice fringes indicative of stacks of polymeric chains on a nanoscale level (FIG. 2B). While ZnCar-MOF is a 3D MOF, ZnCar-CP (molecular model) consists of Zn cations and carnosine ligands linked in a continuous 1-dimensional polymeric chain. Solvent molecules (like water) were not modeled and are expected to occupy leftover coordination sites of Zn cations in ZnCar-CP structure. The bulk material of ZnCar-CP is composed of numerous polymeric chains stacking next to each other, allowing for solvent or drug entrapment between neighboring chains.

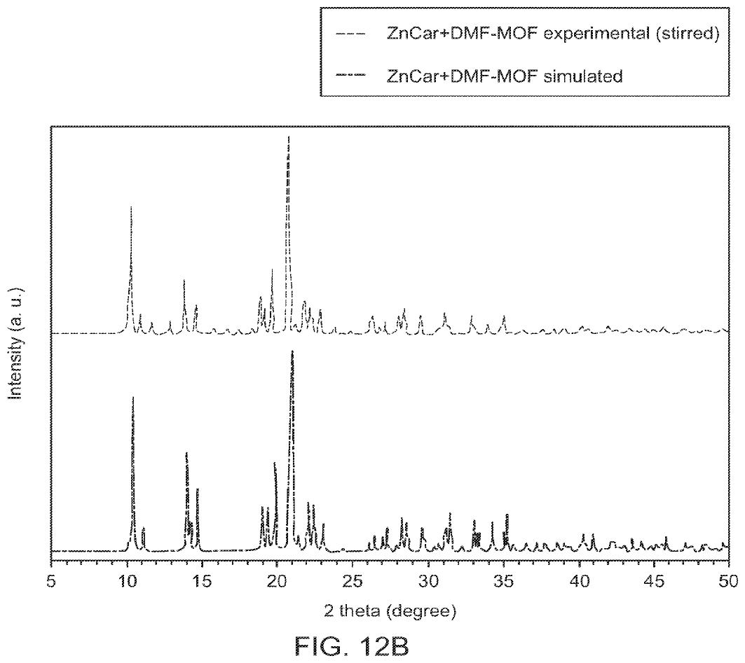

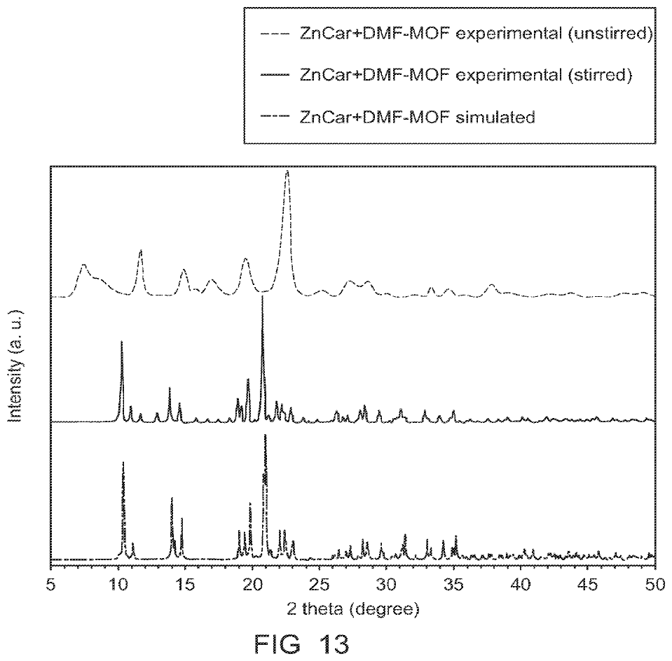

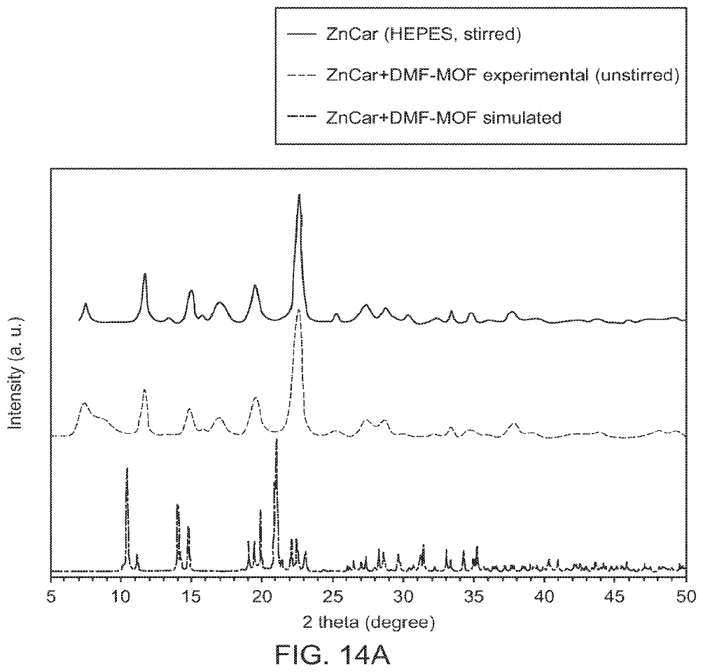

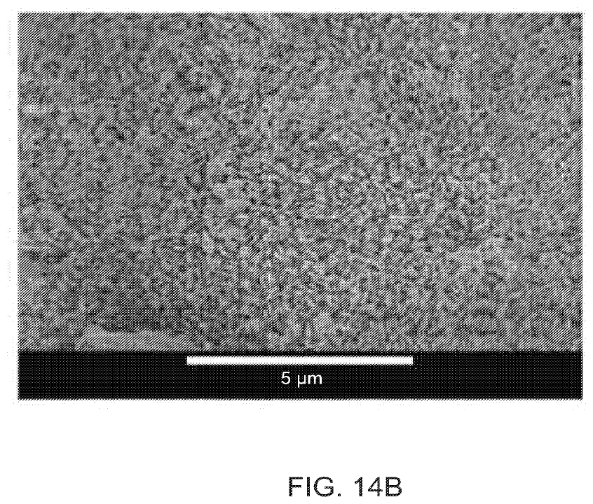

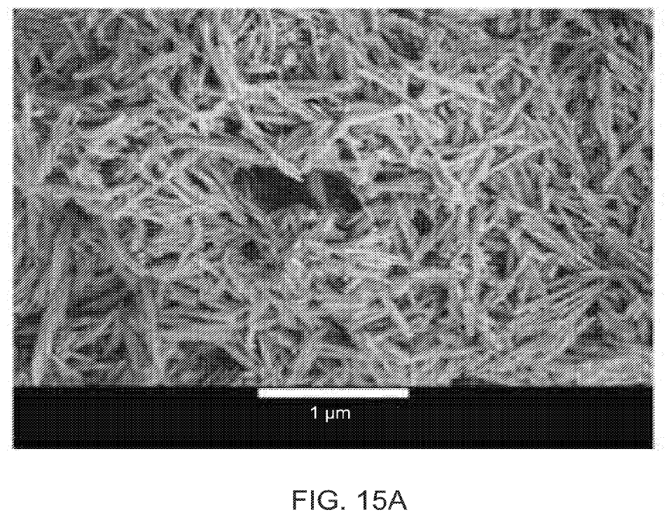

ZnCar-CP and ZnCar-MOF are closely related structures, and it appears that either/or could be formed depending on slight variations in the synthesis conditions. For example, when synthesis is performed in the mixture of HEPES buffer and ethanol, PXRD of the obtained material matches PXRD of ZnCar-MOF, while synthesis in just HEPES buffer results in the formation of ZnCar (FIG. 11), which is hypothesized to have a ZnCar-CP structure. Additionally, when ZnCar-MOF synthesis procedure (see Katsoulidis, A. P.; Park, K. S.; Antypov, D.; Marti-Gastaldo, C.; Miller, G. J.; Warren, J. E.; Robertson, C. M.; Blanc, F.; Darling, G. R.; Berry, N. G., Guest-Adaptable and Water-Stable Peptide-Based Porous Materials by Imidazolate Side Chain Control. Angewandte Chemie 2014, 126 (1), 197-202) is followed and reaction mixture is stirred, ZnCar-MOF is obtained (FIG. 12A and FIG. 12B); however, when the same procedure is followed but the reaction mixture is not stirred, the PXRD of the obtained material matches the PXRD of ZnCar and not of ZnCar-MOF (FIG. 13, FIG. 14A, and FIG. 14B).

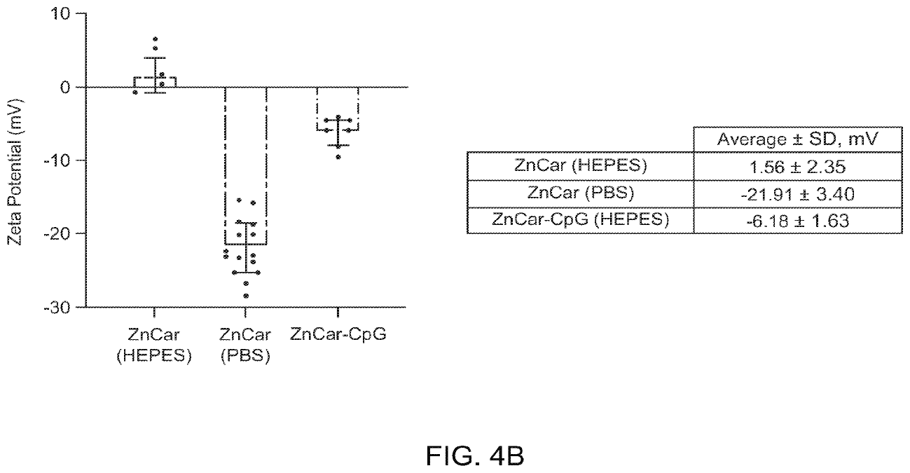

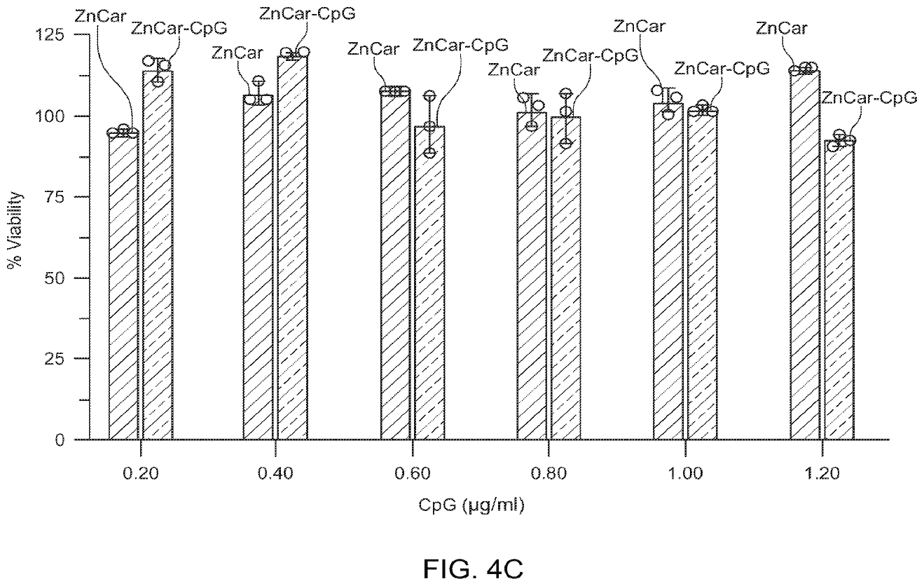

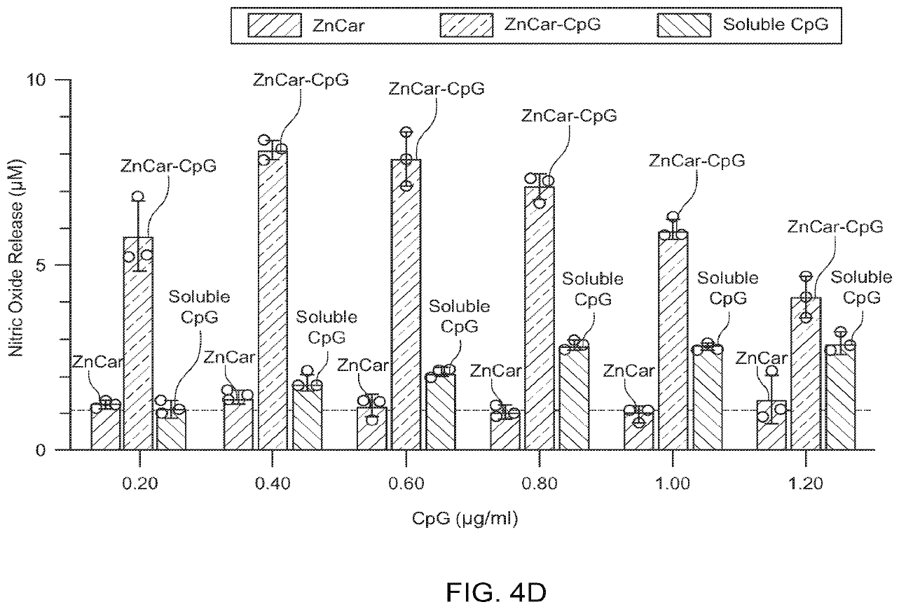

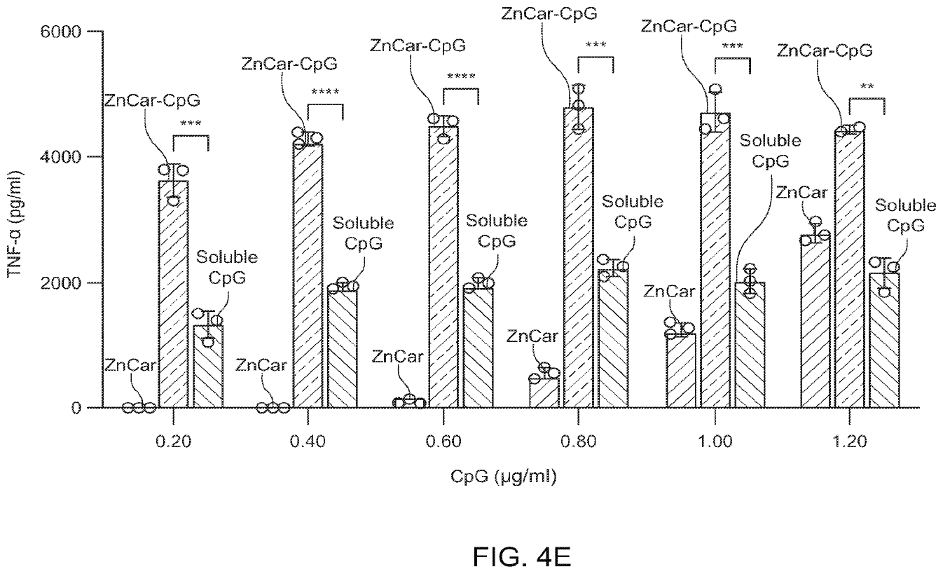

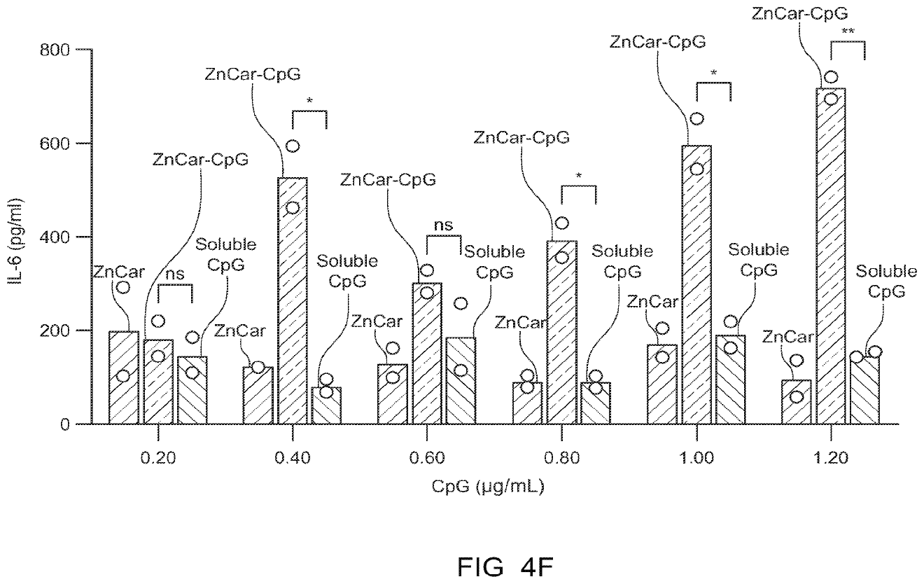

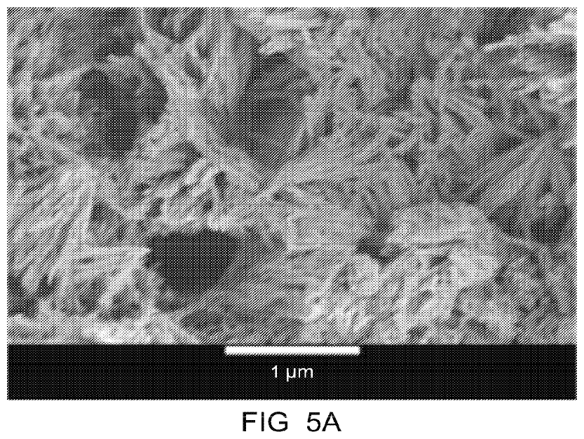

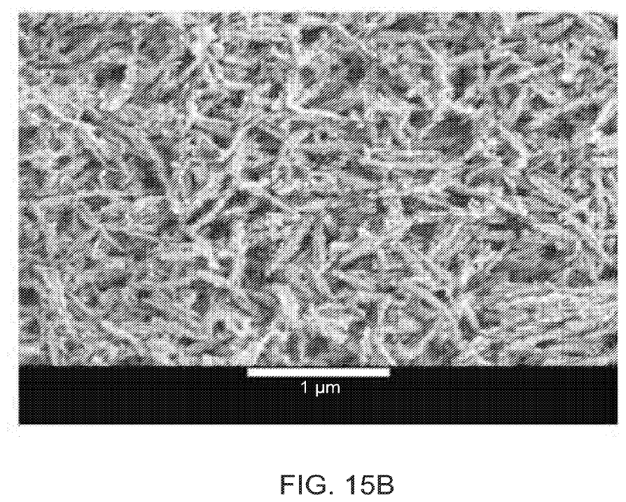

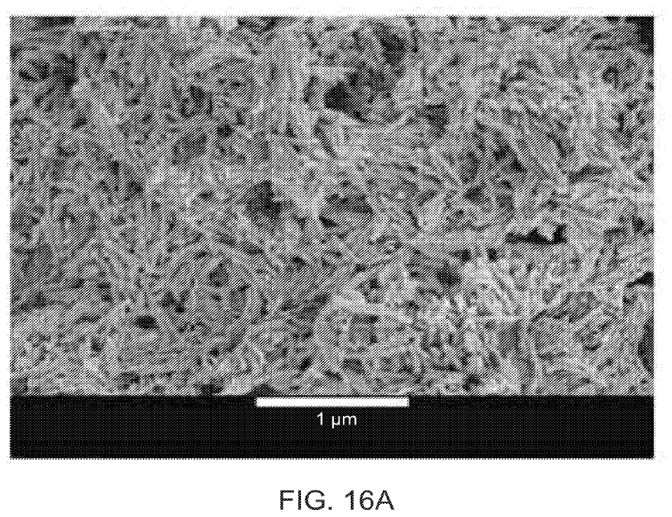

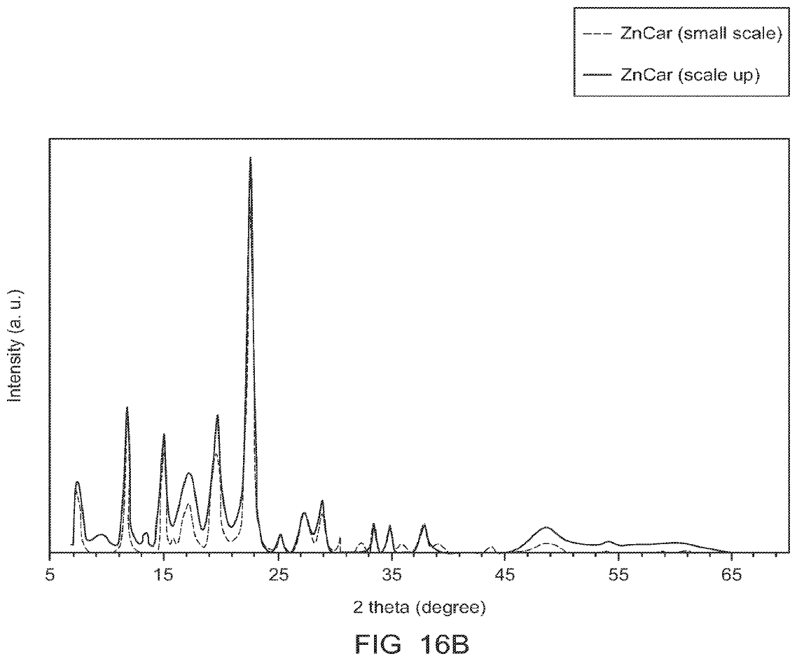

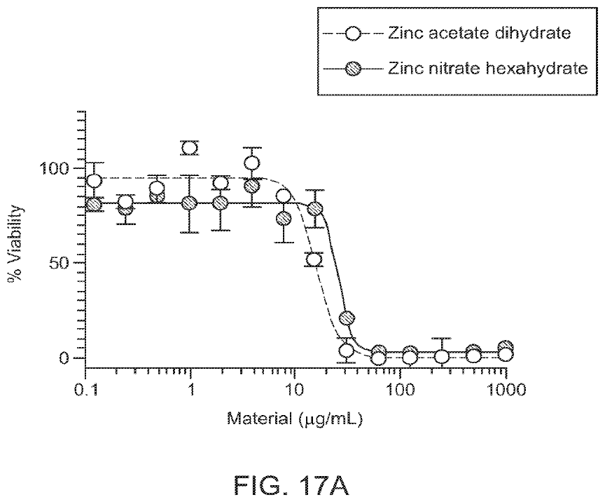

To further evaluate the suitability of ZnCar for biological applications, additional characterization studies were performed. The stability of ZnCar in HEPES and PBS buffer was evaluated at neutral pH. The zeta potential of the ZnCar complexes in HEPES was observed near neutrality (1.56±2.35 mV) whereas ZnCar incubated in PBS had a significantly more negative surface charge (−21.91±3.40 mV) (FIG. 4B). This was likely due to phosphate deposition on the ZnCar surface, as illustrated by apparent crystal growth on the materials surface (FIG. 15A and FIG. 15B). The scalability of ZnCar synthesis was also investigated. Synthesis was performed at a 900 mL scale (60× scale up) in otherwise identical conditions (in 0.1M HEPES at neutral pH with overnight stirring at 37° C.). The resulting ZnCar had similar morphology and crystallinity, determined by SEM and PXRD, respectively, as ZnCar synthesized at smaller scale (FIG. 16A and FIG. 16B). In addition, upon scale up, the reaction yield was increased from ˜50% to ˜100% yield.

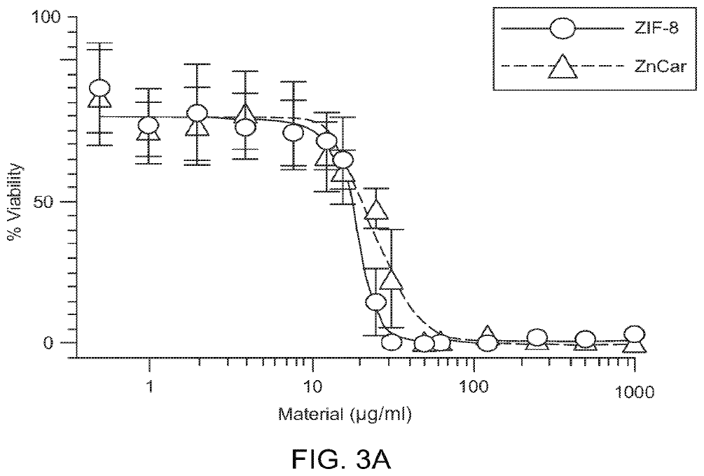

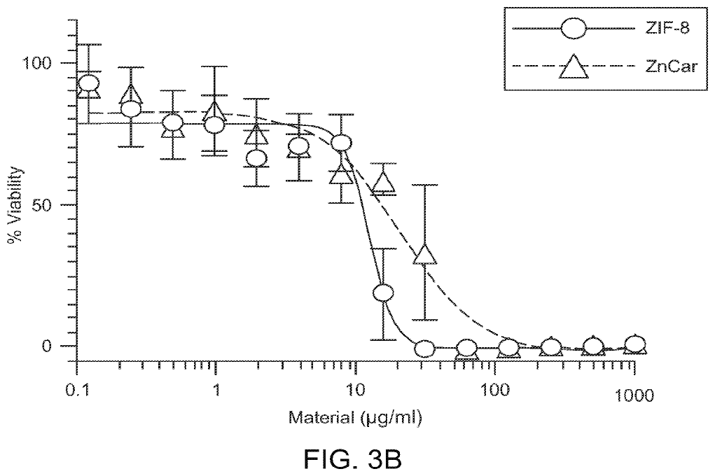

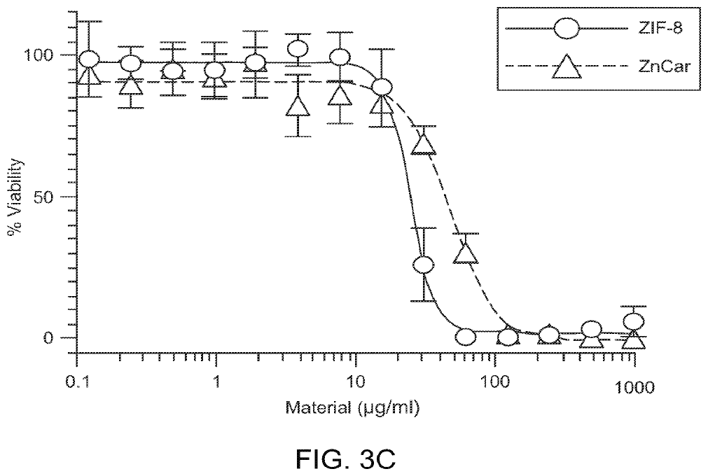

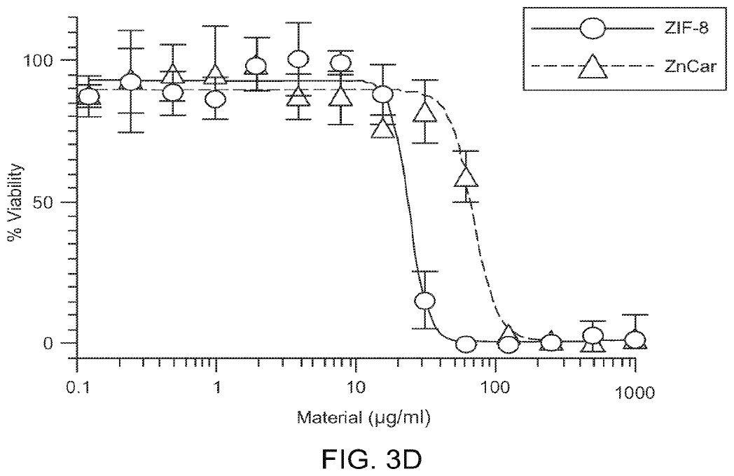

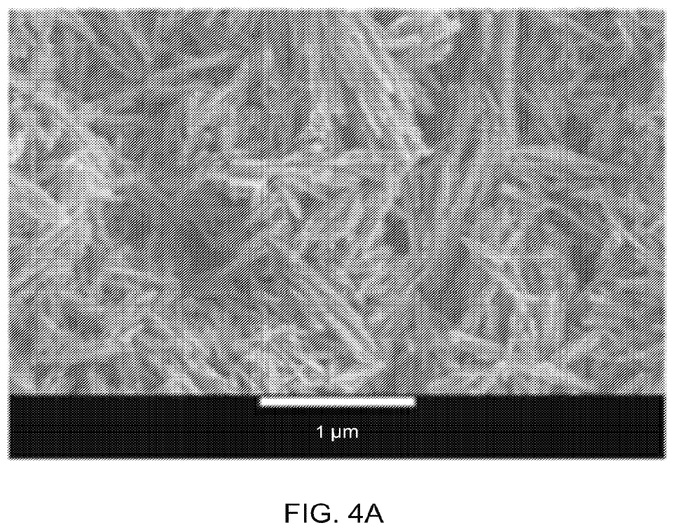

In vitro cytocompatibility of ZnCar was then evaluated with fibroblasts and bone-marrow derived dendritic cells (BMDCs). The determined 50% inhibitory concentrations (IC₅₀) of ZnCar were compared to IC₅₀ of a zeolitic imidazolate framework (ZIF-8). ZIFs are one of the most extensively explored metal-organic materials and ZIF-8 has been previously applied as a drug delivery vehicle for cancer and infectious disease vaccines. Studies indicate that in both fibroblasts and BMDCs, ZnCar was significantly less toxic than ZIF-8 at both 24 and 48 hours. The reduced cytotoxicity compared to ZIF-8 was especially pronounced in BMDCs relative to non-phagocytic fibroblasts (FIG. 3A, FIG. 3B, FIG. 3C, and FIG. 3D; Table 3).

TABLE 3
50% inhibitory concentration (IC₅₀) of ZIF-8 and ZnCar.

| | 50% Inhibitory Concentration (IC₅₀; μg/mL) | | | |
|---|---|---|---|---|
| | ZIF-8 | | ZnCar | |
| Time (h) | 24 | 48 | 24 | 48 |
| Fibroblasts | 19.3 ± 0.6 | 12.5 ± 0.6 | 24.6 ± 1.0 | 20.9 ± 2.1 |
| Dendritic Cells | 25.0 ± 1.0 | 24.2 ± 1.3 | 48.6 ± 2.2 | 69.8 ± 4.2 |

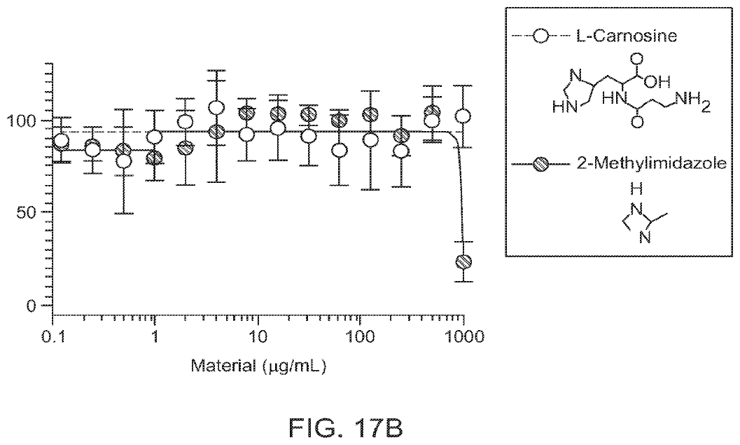

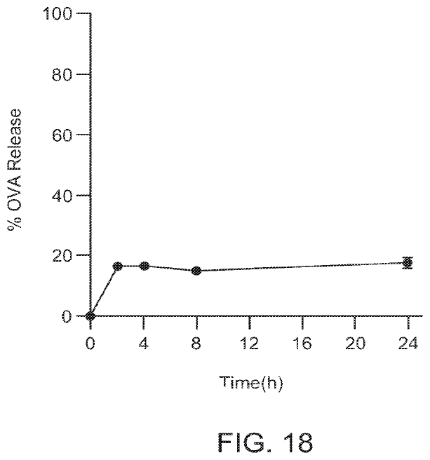

The cytotoxicity in BMDCs is important, as dendritic cells are professional antigen presenting cells (APCs) and therefore are vital in coordinating vaccine responses to protein antigens. The IC₅₀s of each of the reagents used to form the materials were determined in fibroblasts and were much lower for the zinc salts relative to the ligands, indicating that the cytotoxicity of these materials was primarily driven by zinc metal (FIGS. 17A and 17B, and Table 4).

TABLE 4
50% inhibitory concentration (IC₅₀) of Metal Salts and Ligands

| | 50% Inhibitory Concentration (IC50; μg/mL) | | | |
|---|---|---|---|---|
| | Material | | | |
| | Metal salts | | | |
| | zinc | | Ligands | |
| | acetate | zinc nitrate | L- | 2- |
| | dihydrate | hexahydrate | carnosine | methylimidazole |
| Time (h) | 24 | 24 | 24 | 24 |
| fibroblasts | 16.0 ± 1.0 | 25.8 ± 2.1 | >1000 | ~1000 |

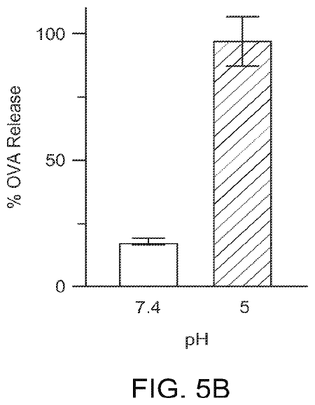

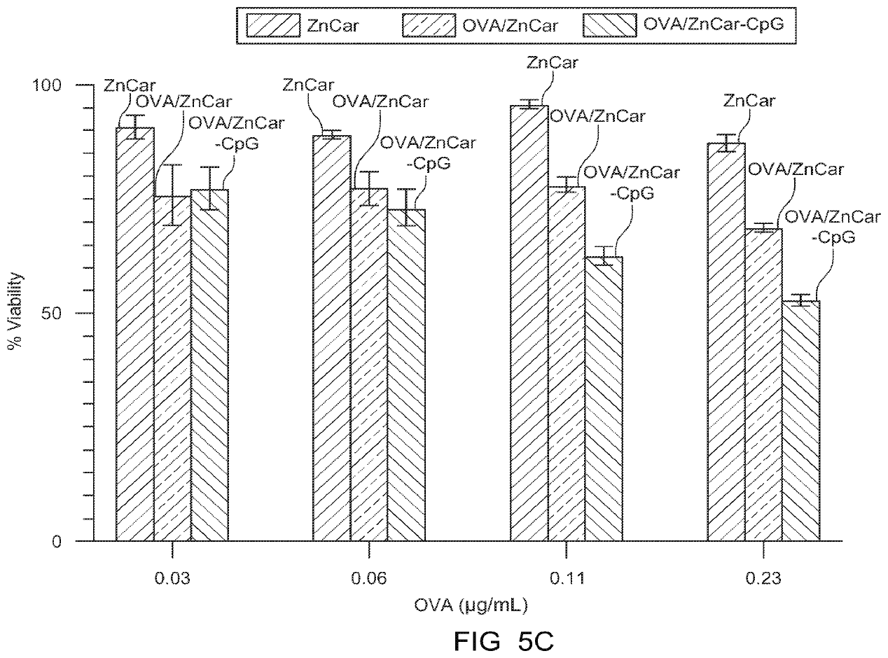

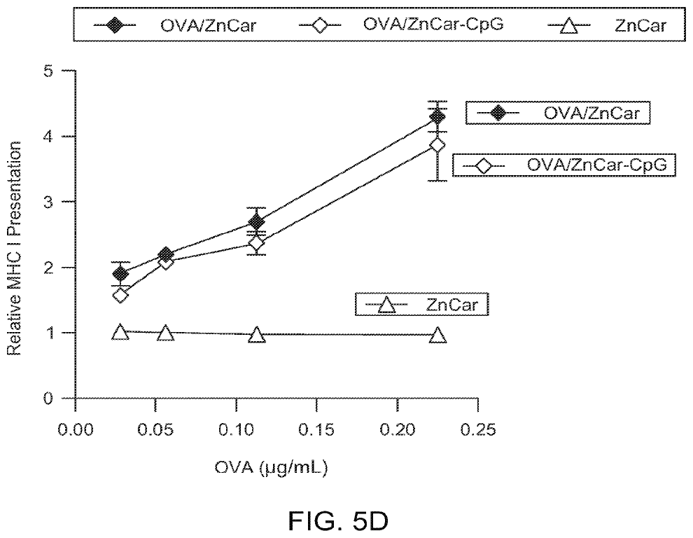

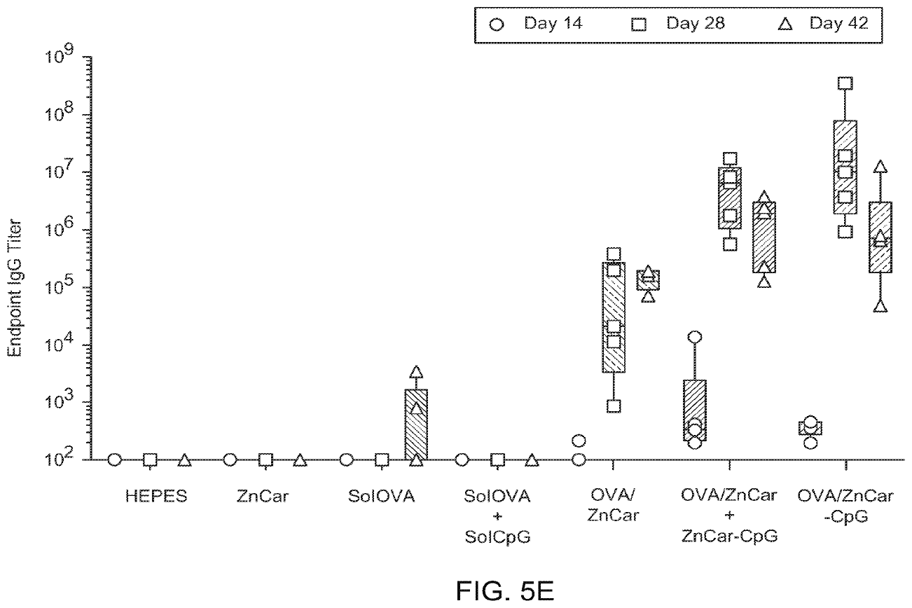

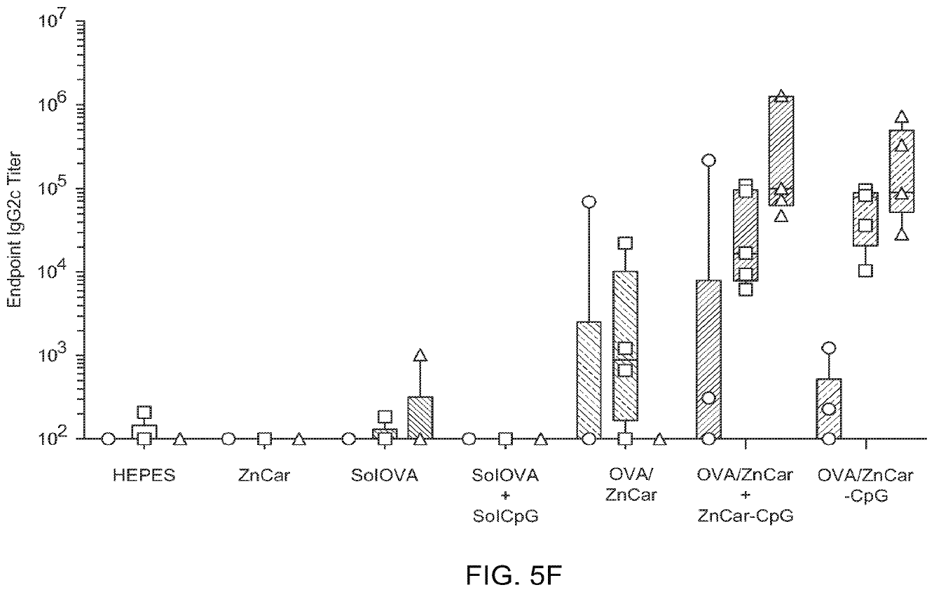

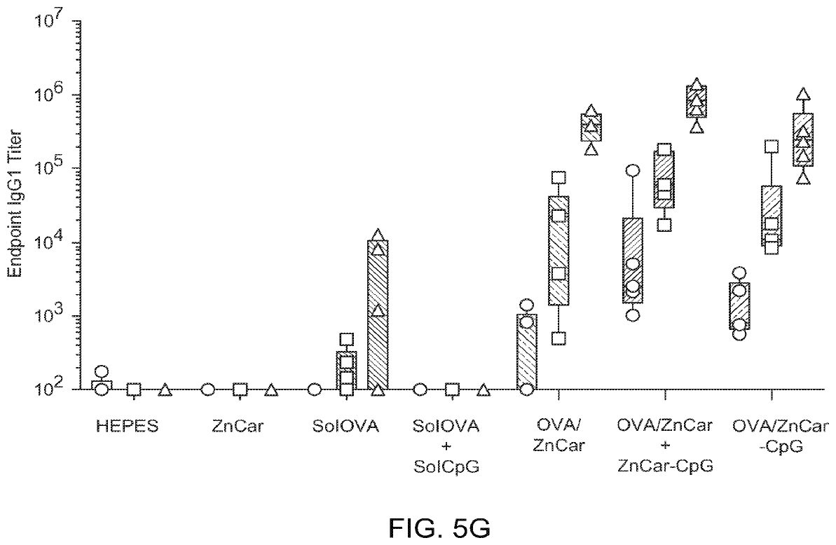

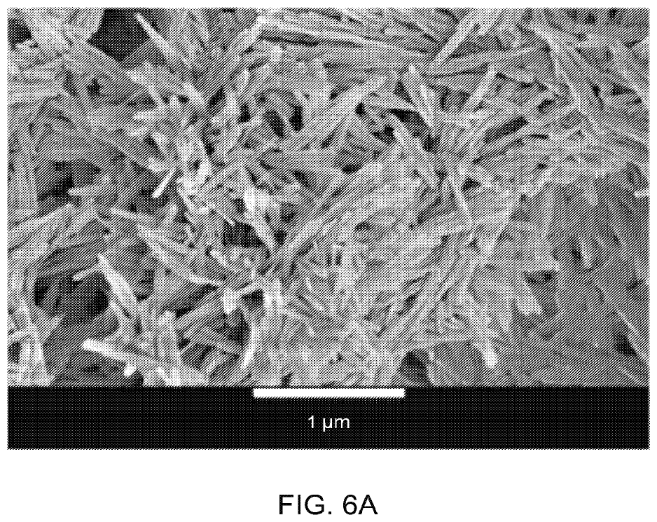

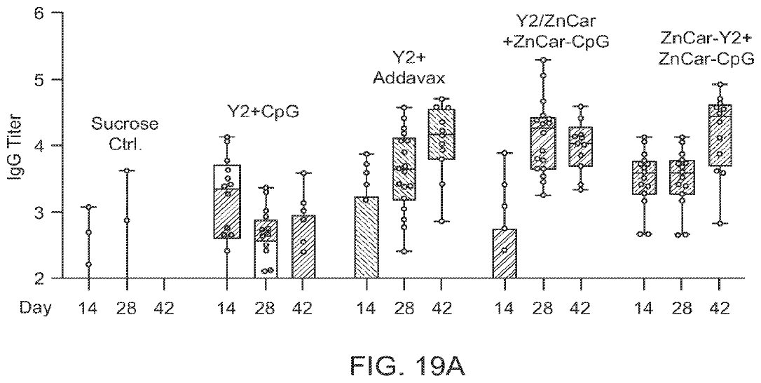

To evaluate ZnCar as vaccine adjuvant, protein antigen and adjuvant formulations with ZnCar were evaluated. The model antigen OVA was added to the solution during the formation of ZnCar (FIG. 5A) to encapsulate the protein within the coordination polymer. Loading of OVA was observed to be 4.3 μg/mg. The release of OVA from the OVA/ZnCar composite at neutral pH illustrated a burst of approximately 18% and then a sustained release for at least 24 hours (FIG. 18). Further, the release was shown to be acid sensitive, with approximately 20% released after 24 hours in pH 7.4 HEPES buffer, but nearly 100% released in pH 5 buffer (FIG. 5B). To incorporate adjuvant, CpG was electrostatically adsorbed on the surface of the material (ZnCar-CpG; FIG. 4A). The final loading quantified for CpG on the ZnCar-CpG composite was 52.9 μg/mg material. The addition of negatively charged CpG to ZnCar resulted in a net negative surface charge of −6.18±1.63 mV, compared to the vehicle charge of ZnCar (1.56±2.35 mV) (FIG. 4B). To evaluate the in vitro innate immune activation of ZnCar and ZnCar-CpG, the materials were incubated with macrophages and compared to equal concentrations of soluble CpG. ZnCar-CpG was not significantly more cytotoxic than empty ZnCar after 24 hours (FIG. 4C), however generation of innate signaling mediator nitric oxide was significantly greater in macrophages cultured with ZnCar-CpG compared to soluble CpG or ZnCar alone (FIG. 4D). Treatment of macrophages with ZnCar-CpG also induced significantly greater secretion of inflammatory cytokines interleukin 6 (IL-6) and tumor necrosis factor alpha (TNF-α) after 24 hours compared to soluble CpG or ZnCar alone (FIG. 4E and FIG. 4F). This indicates a potential for CpG adsorbed to ZnCar material to induce the innate immune responses and the potential for dose sparing of adjuvant when delivered as a ZnCar-CpG composite.

It has been shown that delivery of protein antigen and adjuvant in separate particles can invoke increased vaccine responses. To evaluate this and the humoral response of OVA and CpG formulated with ZnCar, OVA was encapsulated in ZnCar (OVA/ZnCar) and administered to C57Bl/6 mice on day 0 and 21 by intramuscular (IM) injection with CpG co-formulated on the same composite (OVA/ZnCar-CpG) or delivered simultaneously in separately generated materials (OVA/ZnCar+ZnCar-CpG). Total IgG (FIG. 5E), IgG2c (FIG. 5F), and IgG1 (FIG. 5G) serum antibody levels at day 14, 28, and 42 indicate that the use of ZnCar as delivery vehicle resulted in greater total IgG and IgG1 antibody production by day 42 compared to soluble OVA and soluble CpG (solOVA+solCpG). However, the presence of CpG was required to generate Th1 skewed IgG2c response, with the OVA/ZnCar-CpG and OVA/ZnCar+ZnCar-CpG treated mice generating significantly greater antibody titers at all time points compared to all other groups. This data aligns with the in vitro data (FIGS. 4E and 4F) to show that CpG-loaded ZnCar material can stimulate production of cytokines indicative of an innate immune response. Th1 responses are needed for protection against intracellular pathogens like viruses, and some bacteria, fungi, and parasites, and a Th1 response is a desirable outcome for vaccines. Many current vaccines are formulated with the Th2 skewing adjuvant alum (e.g., Biothrax, DTaP, MenB, Gardasil). Further, this illustrates that OVA-loaded ZnCar with and without CpG can generate significant antigen-specific humoral responses. Previous studies with OVA and CpG-loaded ZIF-8 MOF also illustrate significantly higher total IgG titers over soluble OVA and CpG, however, data indicates that ZnCar material has reduced cytotoxicity compared to ZIF-8 MOF (FIG. 3), which could help to limit vaccine side effects.

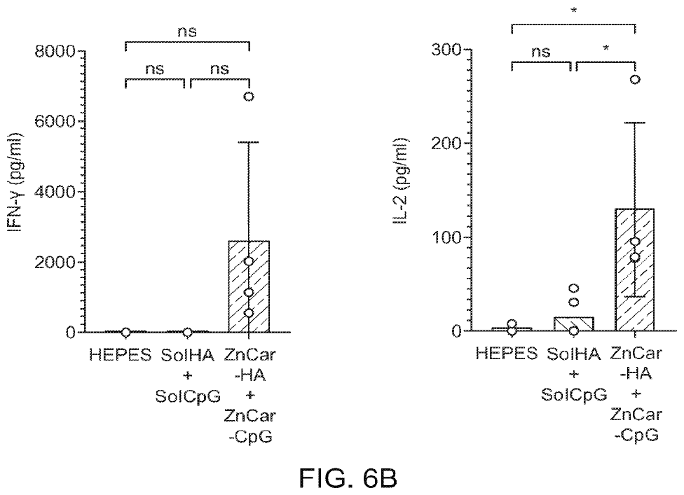

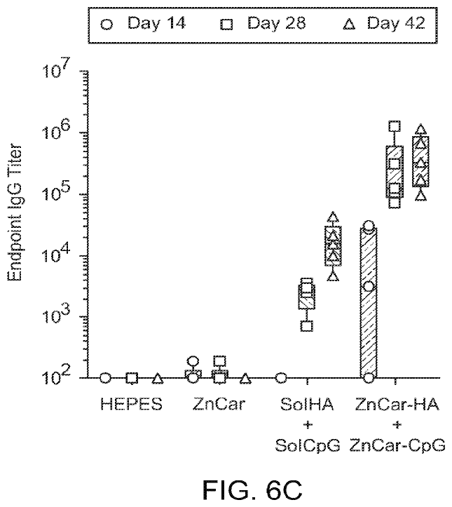

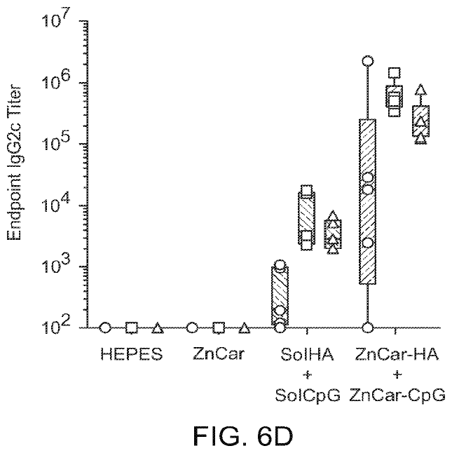

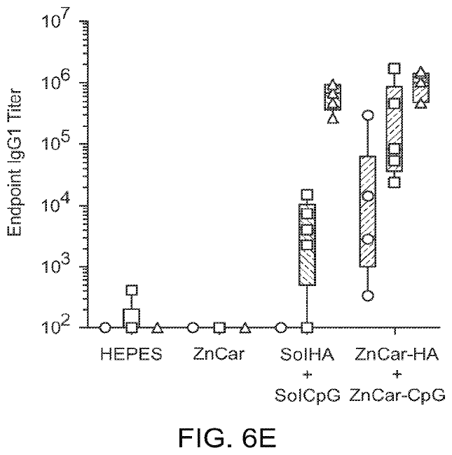

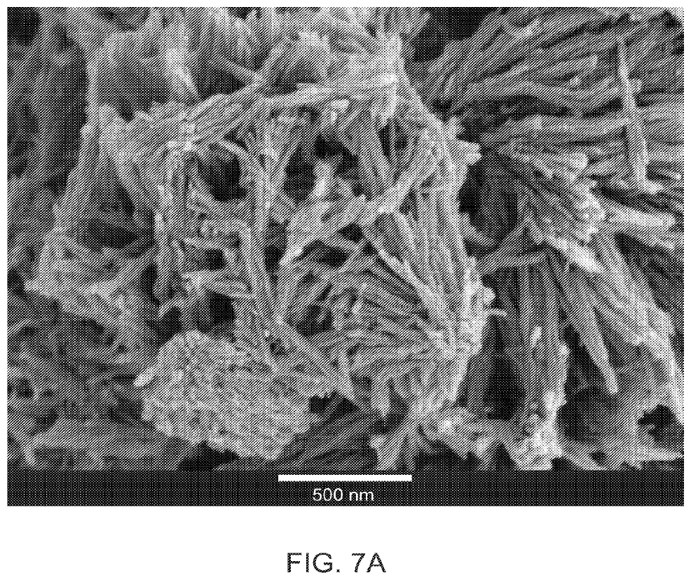

Next, ZnCar was assessed for use as an adjuvant with COBRA H1 HA (HA). His-tagged HA was employed, as this tag facilitated complexation with Zn ions on the surface of ZnCar since the His-tag has affinity for Zinc. HA loading was determined to be much higher than that of OVA (which lacks His-tag) at 45.6 μg/mg material. When HA-loaded ZnCar (FIG. 6A) was used to vaccinate C57BL/6 mice in a prime-boost schedule, again the group with CpG co-delivered in separate vehicles had significantly greater antibody production than control groups (FIG. 6C, FIG. 6D and FIG. 6E).

The efficacy of antigen presentation from BMDCs to T cells was evaluated in vitro using the B3Z T cell clone (FIG. 5D) at non-cytotoxic concentrations (FIG. 5C). Results indicate that ZnCar encapsulating OVA with or without CpG (OVA/ZnCar-CpG and OVA/ZnCar) significantly enhanced antigen cross-presentation relative to the soluble antigen. This could be a function of particle delivery of the antigen as well as the acid-sensitivity of the complex. To evaluate the cellular response in vivo, splenocytes were isolated from HA-vaccinated mice and cultured in the presence of HA. In the supernatant of these restimulated splenocytes, no significant difference in interferon gamma (IFN-7) secretion was observed, but IL-2 secretion was significantly greater in the ZnCar group compared to soluble antigen and adjuvant, indicating a greater degree of antigen-specific T cell response. These findings support previous studies which indicate that vaccination with ZIF-8 MOFs associated with OVA and CpG lead to increased proliferation of T cells as well as pro-inflammatory cytokines with antigen restimulation.

Overall, the formation of a metal-organic coordination material comprised of Zn and carnosine is demonstrated. With the support of molecular modeling and cryo-EM, it is hypothesized that the ZnCar material is a 1D coordination polymer rather than a 3D ZnCar-MOF reported in literature (see Katsoulidis et al.). Infinite coordination polymers exhibit rapid stimulus-dependent depolymerization, a property which may make them more desirable than MOFs for stimulus-dependent drug release applications. ZnCar consists of micron-size fibers that can encapsulate protein antigens as well as associate protein antigens through a His-tag and CpG to the surface. ZnCar exhibited reduced cytotoxicity compared to ZIF-8 MOF in vitro. A dose sparing response was observed for adjuvant CpG wherein less CpG was needed to stimulate innate immune activity in the form of ZnCar-CpG than soluble CpG in vitro. When ZnCar was evaluated in vivo as a vaccine adjuvant, whether formulated with OVA or COBRA H1 HA with CpG, a significantly greater Th1 humoral response was observed. Moreover, cytokine generation with antigen recall indicates an antigen-specific cellular response. These results illustrate the potency of ZnCar metal-organic coordination material in generating antigen-specific humoral and cellular responses when formulated with recombinant protein antigens.

### Example 2—ZnCar Microparticles Enhancing Immune Response Against Influenza

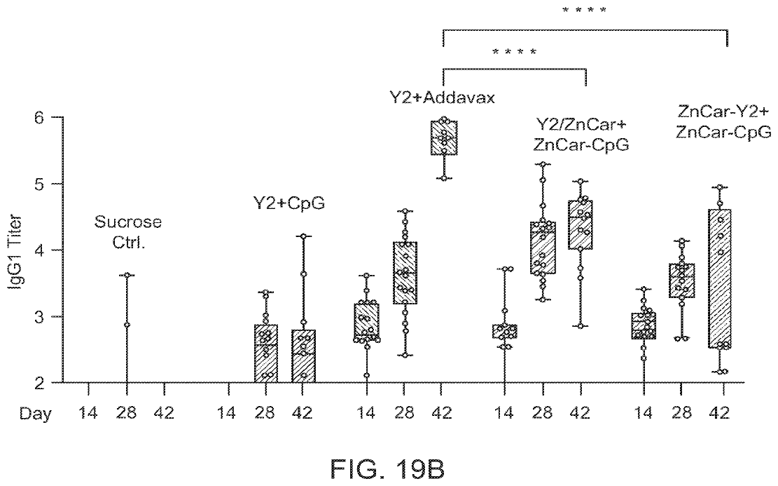

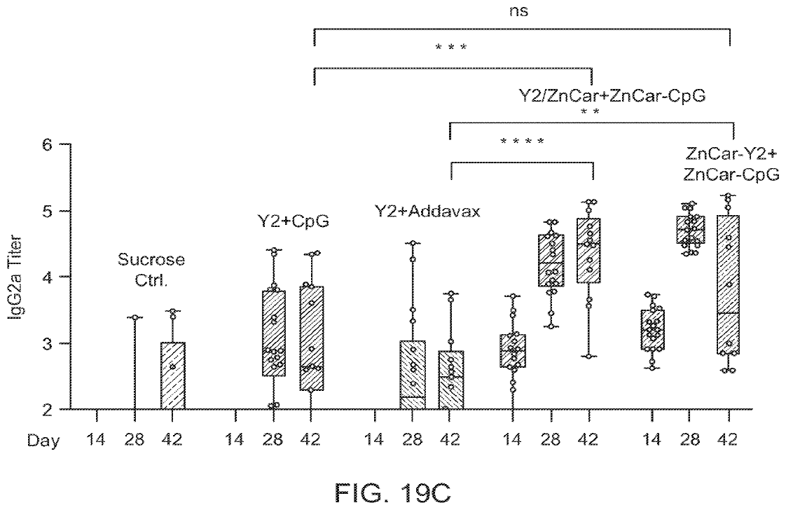

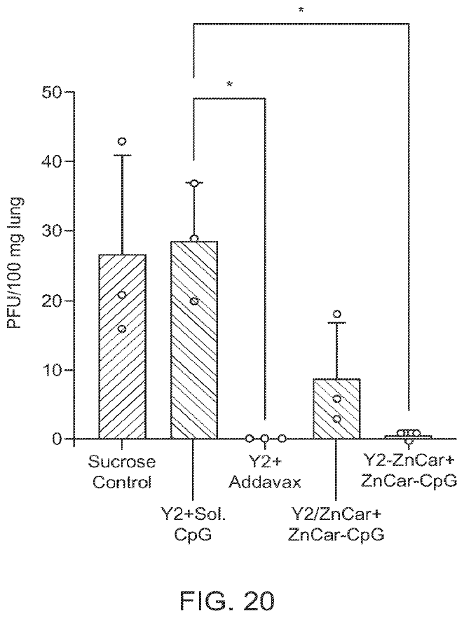

How the zinc carnosine (ZnCar) microparticles (MPs) work in immunizing against influenza was investigated as described herein. In the data shown in FIG. 19A, FIG. 19B, and FIG. 19C, mice were immunized with the hemagglutinin protein Y2 protein. The Y2 protein is a protein designed by the computationally optimized broadly reactive antigen (COBRA) technology that is specific for H1 strains of influenza. Y2/ZnCar denotes protein encapsulated in the ZnCar particles while ZnCar-Y2 are Y2 proteins that complex the protein on the outside of the ZnCar MPs through the His-tag that is present on the Y2 protein. For FIG. 19A, FIG. 19B, and FIG. 19C, BALB/cJ mice (n=18) were vaccinated on a prime+boost schedule (day 0 and day 21). Sera was collected on days 14, 28, and 42 for the quantification of anti-Y2 antibodies via ELISA. As seen in FIG. 19C, the CD4+ T-cell 1 (Th1) antibody, IgG2a was higher in mice vaccinated with ZnCar MPs compared to CpG alone. This indicates that it is inducing a stronger cellular response than CpG alone. There were no significant differences in the total IgG response (FIG. 19A) whether a mouse received Addavax™ (positive clinically relevant control comparable to MF59) or ZnCar-CpG. However, ZnCar-CpG vaccinated mice produced a significantly higher IgG2a titer (FIG. 19C) when compared to the Addavax™ treated mice demonstrating a stronger Th1 response.

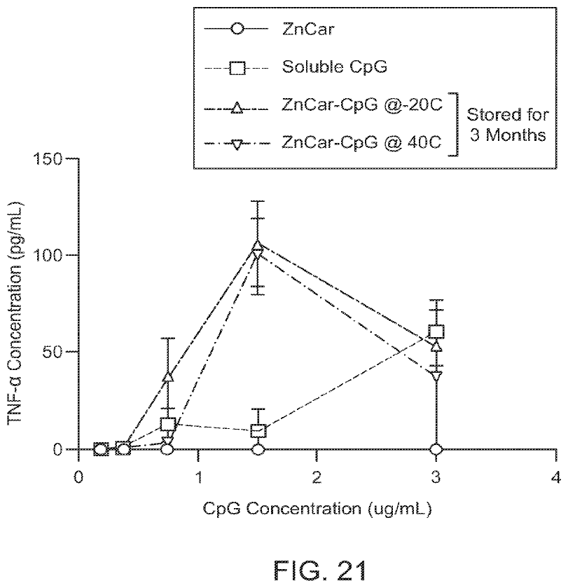

In FIG. 20, mice were immunized with the ZnCar MPs and then challenged with the live virus A/California/07/2009 (H1N1). Three days after challenge with the virus, the viral titer in the lungs of the mice was looked at as an indicator of protection. Mice that were immunized with Y2 complexed on the outside of the ZnCar MPs (Y2-ZnCar) had a statistically significant decrease in the level of virus in the lungs.

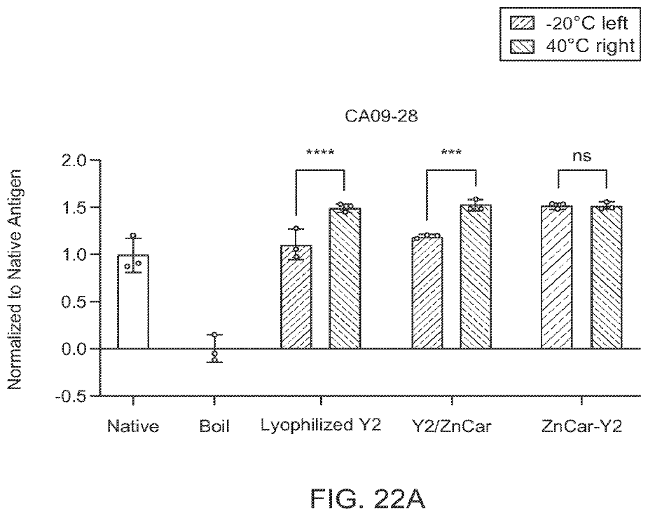

With vaccines there is a need of developing the vaccines that can handle storage outside of the cold chain storage. It was surprising to see that the ZnCar MPs were able to increase the stability of the adjuvant and protein that is incorporated into the MPs. FIG. 21 shows the results of measuring the in vitro activity of ZnCar-CpG after three-months storage at −20° C. or 40° C. In FIG. 21 dendritic cells were cultured with CpG complexed with the ZnCar MPs. Even though the CpG was left at 40° C. for three months, the ZnCar MPs were able to still stimulate DCs similarly to ZnCar MPs that were stored properly in a −20° C. freezer as measured by the production of the inflammatory cytokine TNF-α.

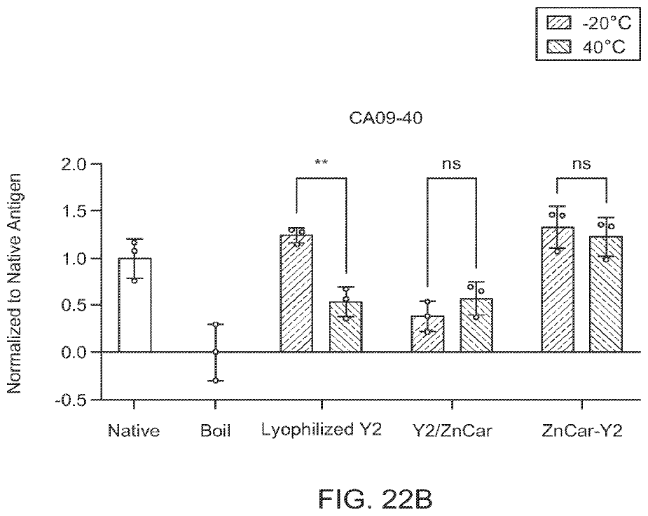

Then, it was explored whether ZnCar MPs were able to keep protein stable. In FIGS. 22A-B a monoclonal antibody that is able to detect the 3-dimensional structure of the Y2 protein was used. Specifically, to determine whether the structure of Y2 was intact after 3 months storage an antibody stability assay was used. CA09-28 (FIG. 22A) or CA09-40 (FIG. 22B) was used to bind a unique epitope on Y2. The assay revealed that ZnCar-Y2 behaved most closely to native antigen whether stored at −20° C. or 40° C.

As can be seen in FIGS. 22A-B, boiled protein results in a complete destruction of the protein. Additionally, lyophilized protein decreases in activity when stored at 40° C., as detected by CA09-40 (FIG. 22B). However, when Y2 protein complexes to the outside of ZnCar MPs, there is no destruction of protein when stored at 40° C.

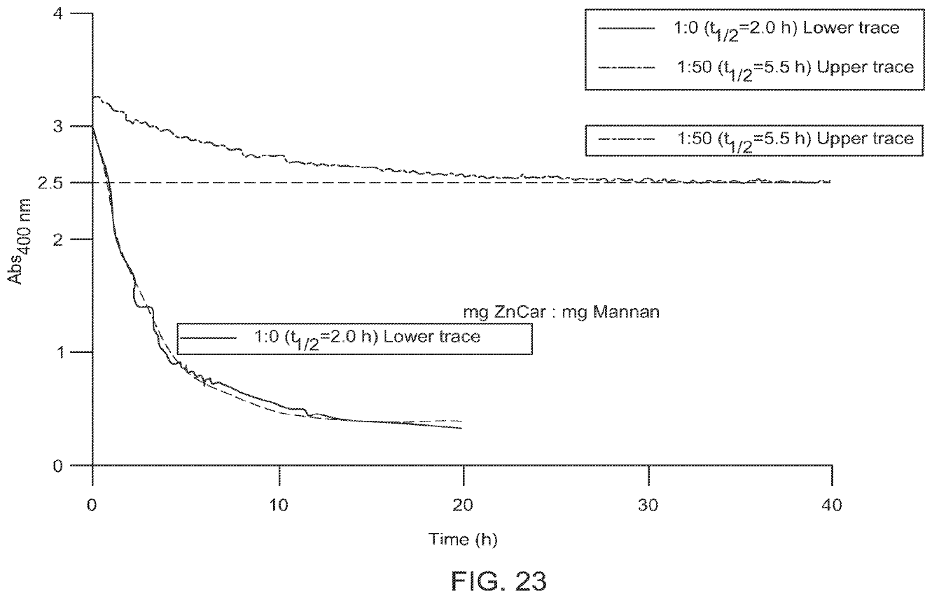

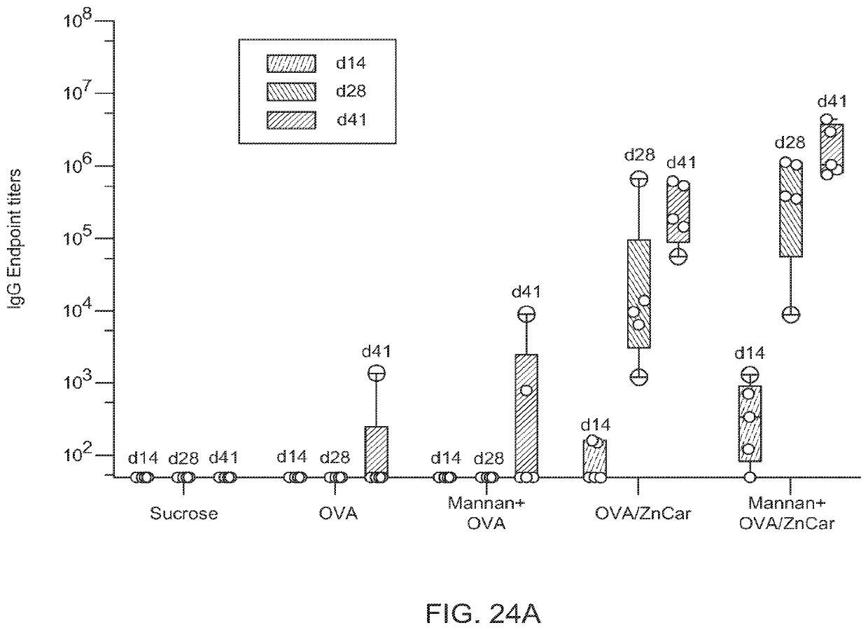

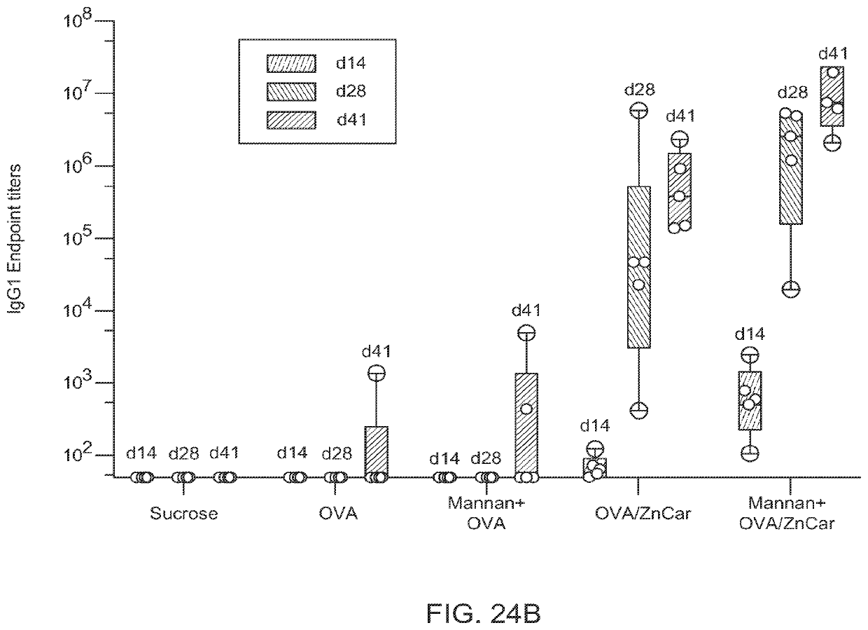

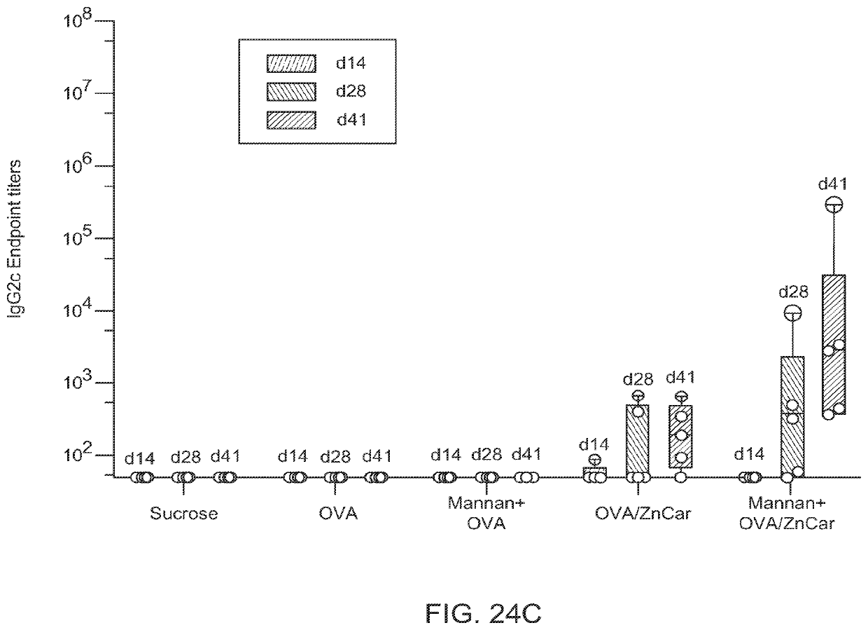

The ZnCar over time does not stay as a homogenous suspension and settles to the bottom of its container. Surprisingly, it was noticed that when ZnCar is mixed with the immunostimulatory polysaccharide, mannan, the ZnCar does not settle down but remains suspended. In FIG. 23, it is shown that ZnCar without mannan settles down relatively quickly (black line, bottom trace) but when mixed with mannan the ZnCar microparticles barely settles down (red line, top trace). Besides allowing the ZnCar to suspend rather well, mannan is also an adjuvant. It was then decided to immunize mice with ZnCar MPs suspended with mannan. As seen in FIG. 24A, FIG. 24B, and FIG. 24C mice were immunized with particles, and IgG (FIG. 24A), IgG1 (FIG. 24B) and IgG2c (FIG. 24C) antibody titers were measured specific for OVA. At day 41 the mannan suspended ZnCar MPs had a statistically highest level of titers for IgG and IgG1 titers. For IgG2c antibodies the titer was also higher, but not statistically significant. This data shows that mannan can increase the suspendability of the ZnCar MPs and can enhance the immune response.

### Example 3—Small Molecule (DOX) in ZnCar

The ability of ZnCar material to serve as a drug delivery vehicle for small molecules was evaluated. A chemotherapeutic doxorubicin (DOX), whose formula is shown below, was chosen as a model small molecule drug:

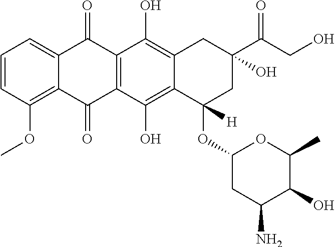 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

It was demonstrated that ZnCar is capable of encapsulating DOX with high efficiency via both encapsulation and surface adsorption routes. It was also demonstrated that DOX remains embedded in the ZnCar material at neutral pH but is efficiently released at lower pH.

### HEPES Buffer

0.1M HEPES solution was prepared by diluting an aliquot of 1M HEPES buffer solution of pH 7.4 (Corning, 25-060-CI; 3 mL) in MilliQ water (27 mL) and adjusting the pH of the resultant solution to 7.4 with 1M NaOH aqueous solution (573 μL).

### Synthesis of DOX/ZnCar (Encapsulation Method).

Doxorubicin hydrochloride (DOX) (0.04 mg for 0.2% mass loading; 0.22 mg for 1% mass loading; 1.25 mg for 5% mass loading; 2.20 mg for 10% mass loading) was dissolved in 2 mL of HEPES buffer. Carnosine (20.53 mg) was dissolved in 2.0 ml of HEPES buffer. Zn(CH₃COO)₂·2H₂O (20 mg) was dissolved in 2 ml of HEPES buffer. Carnosine solution and zinc solution were mixed together (carnosine was added to zinc), and DOX solution was added into obtained mixture. Reaction mixture was stirred at 37° C. for 18 h (except for 10% mass loading, 43 h).

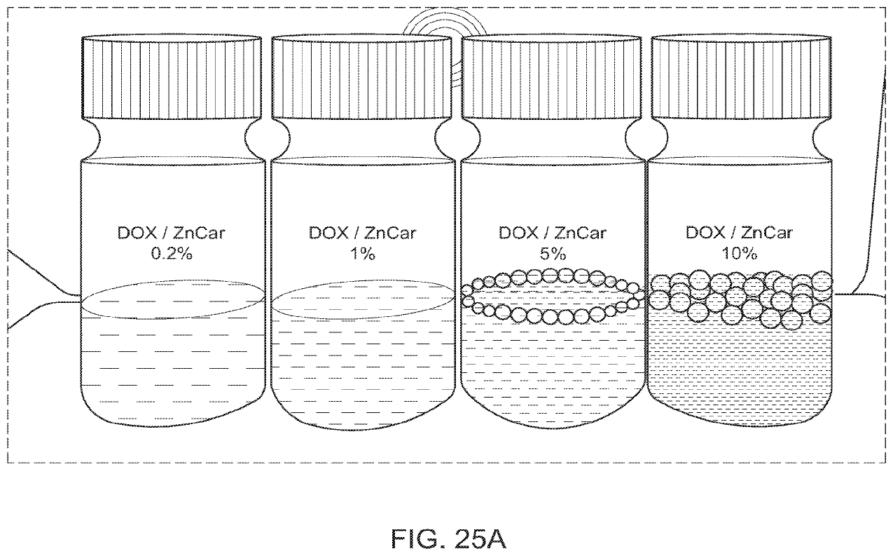

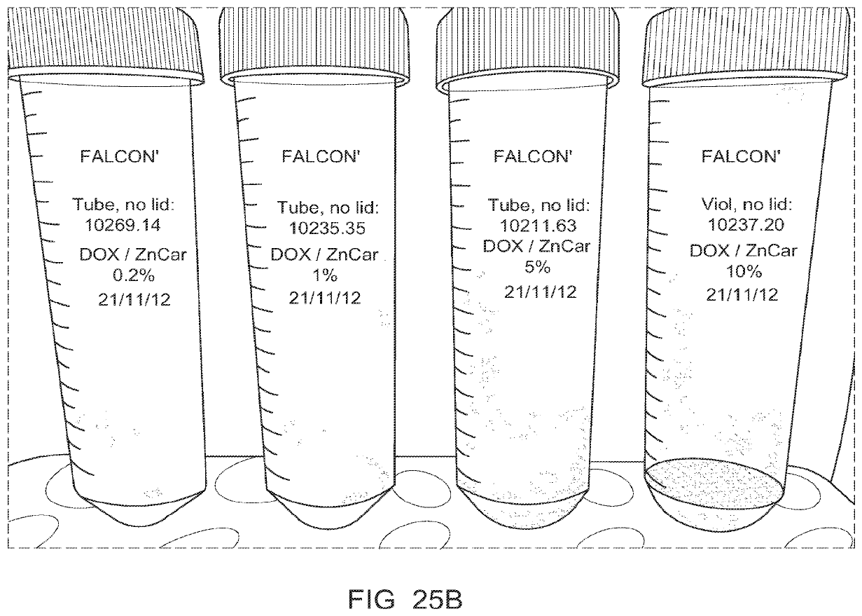

Workup: heating was stopped, and reaction mixtures were cooled down to room temperature. All vials had solids in them (different intensity red color: 0.2% light pink, 1% pink, 5% fuchsia, 10% deep-burgundy; FIG. 25A). Each reaction mixture was transferred into a 50 mL Falcon tube and spun in a centrifuge at 22,000 g at 4 C for 20 min. The supernatant was transferred out. The pellet (different intensity red color: 0.2% light pink, 1% pink, 5% fuchsia, 10% deep-burgundy) was re-suspended in 20 mL of MilliQ water by vortexing then spun in a centrifuge at 22,000 g at 4 C for 20 min. The wash was transferred out and the washing procedure was repeated. Obtained pellet was re-suspended in 5 mL of MilliQ water by vortexing, frozen at −80° C. for 1 h and lyophilized for 2 days. Mass after lyophilizing: 18.5-19.7 mg (FIG. 25B).

### Synthesis of ZnCar-DOX (Surface Adsorption Method).

ZnCar (5.38 mg for 5% mass loading and 5.65 mg for 10% mass loading) was placed in a 2 mL Eppendorf tube and suspended in 200 μL of HEPES buffer using vortex, sonication and pipetting up-and-down. An aliquot of 1 mg/mL DOX solution in HEPES buffer was added to ZnCar suspension (269 μL=0.269 mg for 5% mass loading; 565 μL=0.565 mg for 10% mass loading; also, 296 μL of blank HEPES buffer was added in 5% mass loading tube to keep the final volumes the same for both tubes), and obtained reaction mixture was swirled a few times, vortexed very lightly and placed on a tube rotator. The tube was rotated for 20 h.

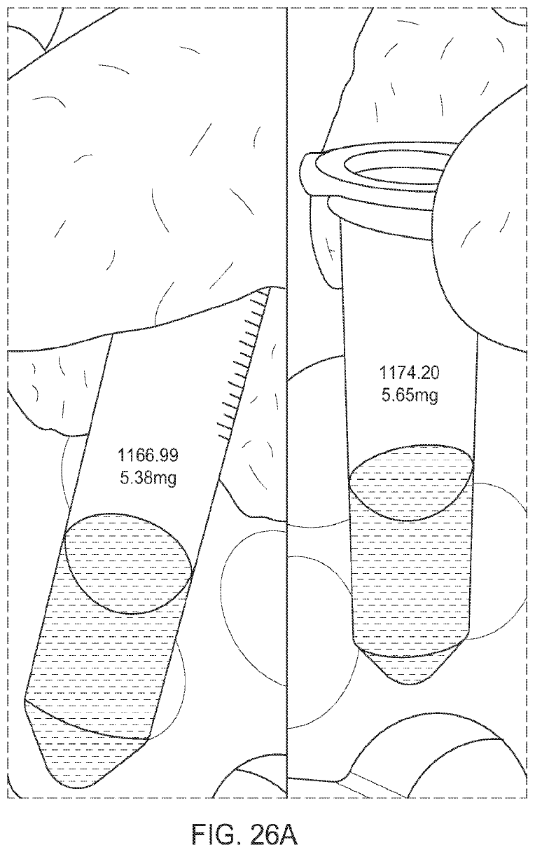

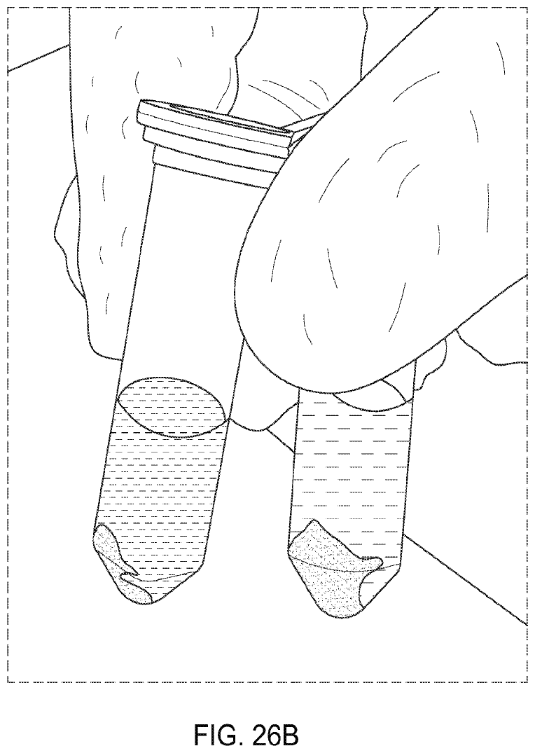

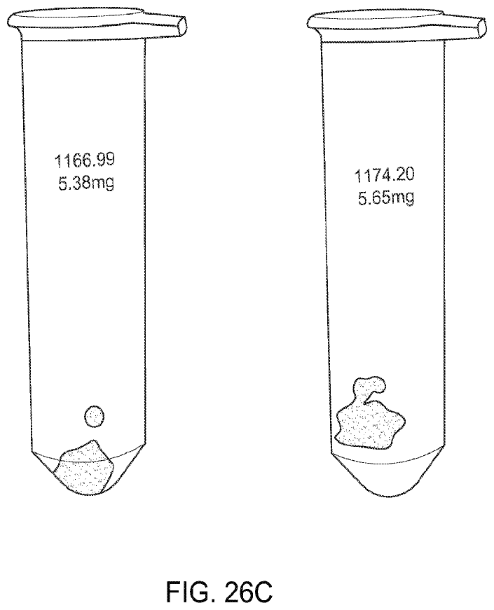

Workup: Rotation was stopped (FIG. 26A), and the tube was spun in a microcentrifuge at 21,100 g at 4 C for 20 min (FIG. 26B). The supernatant was transferred out. The pellet (dark-red) was treated with 600 μL of MilliQ water and re-suspended by pipetting up-and-down. The tube was spun in a microcentrifuge at 21,100 g at 4 C for 20 min. The wash was transferred out and the washing procedure was repeated two more times. The pellet was treated with 600 μL of MilliQ water and re-suspended by pipetting up-and-down. Obtained suspension was frozen at −80° C. for 25 min and lyophilized for 2 days.

Mass after lyophilizing: 4.1-5.2 mg (FIG. 26C).

### DOX Quantification

DOX-loaded materials were decomposed with 0.3 M acetate buffer and obtained solutions were analyzed for DOX absorbance at 480 nm (Table 5).

TABLE 5
DOX quantification results.

| | | | | Encapsulation |
|---|---|---|---|---|
| | | | Detected | efficiency |
| | Theor. | | loading | relative to |
| | loading | Theor. loading | sample | theor. loading |
| | based on | based on | decomposed | based on |
| | projected | experimental | with acetate | experimental |
| | yield | yield | buffer | yield |
| DOX/ZnCar | | | | |
| (encapsulation) | | | | |
| ZnCar, mg | | | | |
| Projected yield: 22 mg | | | | |
| Experimental yield: 19.7 mg | 0.2% | 0.2% | 0.2% | 100% |
| Experimental yield: 18.9 mg | 1.0% | 1.2% | 1.4% | 116% |
| Experimental yield: 18.5 mg | 5.0% | 6.7% | 4.4% | 66% |
| Experimental yield: 19.2 mg | 10.0% | 11.5% | 6.2% | 54% |
| ZnCar-DOX (surface) | | | | |
| Projected yield: 5.38 mg | 5.0% | 6.5% | 3.0% | 46% |
| Experimental yield: 4.12 mg | | | | |
| Projected yield: 5.65 mg | 10.0% | 10.8% | 4.4% | 41% |
| Experimental yield: 5.20 mg | | | | |

### Dox Release Studies

Two buffers were used for the release studies: 0.1M HEPES buffer (pH 7.4) and 0.3 M acetate buffer (pH 5.0). Drug release studies were performed from DOX/ZnCar material (detected DOX mass loading: 4.40%). Each timepoint was done in duplicates. Each DOX/ZnCar replicate was placed in 2 mL Eppendorf tube and treated with one of the buffers at 1 mg/mL concentration. Obtained mixture was incubated at 37° C. for indicated time. After incubation, the tubes with samples were spun down in a microcentrifuge at 21,100 g at 4 C for 20 min and supernatants were measured for absorbance at 480 nm (maximum of absorbance of free DOX).

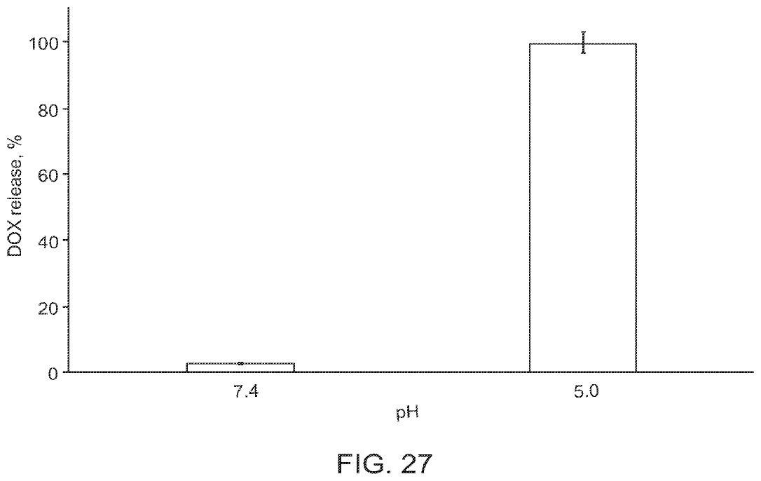

Results for 1 h timepoint are shown below (FIG. 27). Similar results were obtained for other timepoints studied. Very small percentage of DOX was released in HEPES buffer at timepoints 3 h, 5 h, 24 h, and 1 week, and nearly quantitative release of DOX was observed in acetate buffer at timepoints 0.5 h and 2 h.

1. A composite composition comprising:
a metal-organic coordination polymer matrix; and
one or more bioactive compositions carried by the metal-organic coordination polymer matrix, wherein the metal-organic coordination polymer matrix comprises polymeric chains including a repeating unit of formula I: 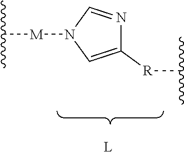
where M is a transition metal, L is a linker including R, wherein R comprises a moiety for coordinating with the transition metal.

2. The composite composition of claim 1, wherein R comprises an oxygen atom for coordinating with the transition metal.

3. The composite composition of claim 2, wherein R is carboxyl.

4. The composite composition of claim 3, wherein the carboxyl and imidazole moieties are provided by histidine or histidine moiety.

5. The composite composition of claim 4, wherein the histidine or histidine moiety forms an amide bond with another amino acid.

6. The composite composition of claim 5, wherein the amino acid is an α-amino acid, β-amino acid, or unnatural amino acid.

7. The composite composition of claim 1, wherein the bioactive composition comprises a small molecule, nucleic acid, or protein.

8. The composite composition of claim 7, wherein the protein is a recombinant protein.

9. The composite composition of claim 1, wherein the bioactive composition is an antigen.

10. The composite composition of claim 1, wherein the bioactive composition comprises one or more pharmaceuticals.

11. The composite composition of claim 1, wherein the bioactive composition is present in an amount of 0.5-70 weight percent of the composite composition.

12. (canceled)

13. The composite composition of claim 1, wherein the polymeric chains are stacked.

14. The composite composition of claim 13, wherein the polymeric chains provide a fibrous morphology.

15. The composite composition of claim 13, wherein the bioactive composition resides in spaces between the polymeric chains.

16. The composite composition of claim 1, further comprising an adjuvant for the bioactive composition, the adjuvant comprising one or more ligands for pattern recognition receptors (PRR) or one or more oligodeoxynucleotides.

17-18. (canceled)

19. The composite composition of claim 16, wherein the adjuvant is adsorbed onto surfaces or inside the metal-organic coordination polymer matrix.

20. The composite composition of claim 16, wherein the adjuvant is present in an amount of 0.01 to 20 weight percent of the composite composition.

21. The composite composition of claim 1, wherein the metal-organic coordination polymer matrix degrades at pH values less than neutral pH to release the bioactive agent.

22. The composite composition of claim 1, wherein the metal-organic coordination polymer matrix degrades under biological conditions.

23. The composite composition of claim 9, wherein the composition is a vaccine.

24. The composite composition of claim 1, wherein the transition metal is first row transition metal selected from the group consisting of zinc, copper, manganese, and cobalt.

25. (canceled)

26. The composite composition of claim 1, wherein the metal-organic coordination polymer matrix is zinc-carnosine coordination polymer.

27. The composite composition of claim 1, wherein the repeating unit comprises zinc, manganese, or copper, and a peptide comprising histidine and at least one additional amino acid.

28. The composite composition of claim 27, wherein the peptide is selected from the group consisting of His-Ala-OH, His-Asp-OH, H-His-Leu-OH, H-His-Tyr-OH, H-Asp(His-OH)—OH, H-Gly-Gly-His-OH, H-Gly-His-Gly-OH, and H-Gly-His-Lys-OH.

29-36. (canceled)

37. The composite composition of claim 1, wherein the repeating unit comprises copper sulfate salt and histamine.

38. The composite composition of claim 1, wherein the one or more bioactive compositions are coupled to the metal-organic coordination polymer matrix via a His-tag.

39. A composition comprising the composite composition of claim 1 suspended in a polysaccharide solution.

40. The composition of claim 39, wherein the polysaccharide solution comprises mannan.

41-49. (canceled)
